(19) United States
(12) Patent Application Publication
(10) Pub. No.: US 2017/0055878 A1
(43) Pub. Date: Mar. 2, 2017

(54) METHOD AND SYSTEM FOR RESPIRATORY MONITORING

(71) Applicant: University of Connecticut, Farmington, CT (US)

(72) Inventors: KI H. CHON, Storrs, CT (US); Bersain A. Reyes, Storrs, CT (US)

(21) Appl. No.: 15/179,925

(22) Filed: Jun. 10, 2016

Related U.S. Application Data
(60) Provisional application No. 62/173,667, filed on Jun. 10, 2015.

(51) Int. Cl. A61B 5/08; A61B 5/091; A61B 5/00; A61B 5/087

(52) U.S. Cl. CPC A61B 5/0816; A61B 5/0873; A61B 5/091; A61B 5/0022

(57) ABSTRACT

A method and corresponding apparatus for monitoring breathing computes a calibration signal from a first sequence of images of a user's chest to produce a calibration model.. The calibration signal is representative of movement of the user's chest during a first time period during which the user is using an incentive spirometer a commercially-available (IS). The first sequence of images corresponds to the first time period. A method and corresponding apparatus employ the calibration model to produce a breathing information estimate about the user's breathing from a second sequence of images of the user's chest corresponding to a second time period during which the user is not using the a commercially-available IS. Example applications for the method and corresponding apparatus include vital sign applications for personalized healthcare through use of a smartphone.

### RELATED APPLICATIONS

This application claims the benefit of U.S. Provisional Application No. 62/173,667, filed on Jun. 10, 2015. The entire teachings of the above application are incorporated herein by reference.

### GOVERNMENT SUPPORT

This invention was made with government support under grant No. W81XWH-12-1-0541 from US Army Medical Research and Material Command (US-AMRMC). The government has certain rights in the invention.

### BACKGROUND

Today's wireless devices, such as smartphones, may be equipped with cameras, a user interface, and powerful processors. A wireless device with such resources can be programmed with a software application (“app”) to configure the device to capture images, analyze the images, and produce analytical results, such as for use in the growing field of personalized health care.

### SUMMARY

In the field of personalized healthcare, two breathing status parameters that may be useful to monitor include tidal volume (V_{T}) and respiration rate (RR). Tidal volume is a lung volume of air moved with each breath. Respiratory rate is a number of breaths per unit of time. Methods for V_{T} and RR measurements have been designed for clinical settings or research centers, and such methods employ specialized devices, such as spirometer devices, that are not translated easily to everyday use due to their high costs, need for skilled operators, or limited mobility. In contrast to a spirometer device, an incentive spirometer (IS) is a low-cost off-the-shelf device that is accessible to and easily used by the general population

Accordingly, a device for monitoring breathing may comprise a processor. The processor may be configured to compute a calibration signal from a first sequence of images of a user's chest to produce a calibration model. The calibration signal may be representative of movement of the user's chest during a first time period during which the user is using an incentive spirometer. The first sequence of images may correspond to the first time period. The processor may be further configured to employ the calibration model to produce a breathing information estimate about the user's breathing from a second sequence of images of the user's chest corresponding to a second time period during which the user is not using the incentive spirometer.

The breathing information estimate may include a representation of tidal volume, respiratory rate, or instantaneous respiratory rate, or a combination thereof.

The device may be a smartphone that includes the processor and an integrated camera configurable to capture the first sequence of images and the second sequence of images.

The smartphone may further include a user interface. The processor may be further configured to output a representation of the breathing information estimate via the user interface. The processor may be further configured to determine the first time period and the second time period based on interactions with the user via the user interface.

The smartphone may further include a network interface. The processor may be further configured to output a representation of the breathing information estimate via the network interface.

The smartphone may further include a hardware interface configured to detect a usage signal. The usage signal may represent usage of the incentive spirometer. The processor may be further configured to determine the first and second time periods based on detection of the usage signal.

The device may be a network server.

The network server may include a network interface. The network server may be configured to receive the first sequence of images and the second sequence of images via the network interface. The processor may be further configured to output a representation of the breathing information estimate via the network interface.

Using the incentive spirometer may include inhaling through the incentive spirometer or exhaling through the incentive spirometer.

The first time period may include at least two time periods during which the user is using the incentive spirometer. The first sequence of images may include at least two sequences of images that may correspond to the at least two time periods. The calibration signal may be further representative of movement of the user's chest during respective periods of the at least two time periods. The user may achieve a different target level on the incentive spirometer during respective periods of the at least two time periods.

The device may further comprise a camera configurable to capture the first sequence of images and the second sequence of images.

The device may further comprise a user interface. The processor may be further configured to output a representation of the breathing information estimate via the user interface.

The device may further comprise a network interface. The processor may be further configured to output a representation of the breathing information estimate via the network interface.

The device may be a component within a system. The system may include the device and a camera configurable to capture the first sequence of images and the second sequence of images.

The calibration model may be a linear model.

According to another embodiment, a method for monitoring breathing may compute a calibration signal from a first sequence of images of a user's chest to produce a calibration model. The calibration signal may be representative of movement of the user's chest during a first time period during which the user is using an incentive spirometer. The first sequence of images may correspond to the first time period. The method may further comprise employing the calibration model to produce a breathing information estimate about the user's breathing from a second sequence of images of the user's chest corresponding to a second time period during which the user is not using the incentive spirometer.

The breathing information estimate may include a representation of tidal volume, respiratory rate, or instantaneous respiratory rate, or a combination thereof.

The method may further comprise capturing the first sequence of images and the second sequence of images by a camera.

The method may further comprise outputting a representation of the breathing information estimate via a user interface or a network interface.

The method may further comprise determining the first time period and the second time period based on interactions with a user via a user interface.

The method may further comprise detecting a usage signal, the usage signal representing usage of the incentive spirometer and determining the first and second time periods based on the usage signal detected.

The method may further comprise receiving the first sequence of images and the second sequence of images via a network interface and outputting a representation of the breathing information estimate via the network interface.

Using the incentive spirometer may include inhaling through the incentive spirometer or exhaling through the incentive spirometer.

The first time period may include at least two time periods during which the user is using the incentive spirometer. The first sequence of images includes at least two sequences of images may correspond to the at least two time periods. The calibration signal may be further representative of movement of the user's chest during the at least two time periods. The user may achieve a different target level on the incentive spirometer during respective periods of the at least two time periods.

The calibration model may be a linear model.

Yet another example embodiment may include a non-transitory computer-readable medium having stored thereon a sequence of instructions which, when loaded and executed by a processor, causes the processor to perform methods disclosed herein.

It should be understood that embodiments disclosed herein can be implemented in the form of a method, apparatus, system, or computer readable medium with program codes embodied thereon.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The foregoing will be apparent from the following more particular description of example embodiments of the invention, as illustrated in the accompanying drawings in which like reference characters refer to the same parts throughout the different views. The drawings are not necessarily to scale, emphasis instead being placed upon illustrating embodiments of the present invention.

FIG. 1A is a block diagram of an example application for which embodiments disclosed herein may be applied.

FIG. 1B is a block diagram of an example embodiment of an incentive spirometer that may be employed within embodiments disclosed herein.

FIG. 2 is a block diagram of an example embodiment of a system.

FIG. 3 is a flow diagram of an example embodiment of a method.

FIG. 4 is a block diagram of plots of signals representative of a user's chest movements.

FIG. 5 is a block diagram of an acquisition setup for a subject breathing through an incentive spirometer.

FIG. 6 includes plots of examples of acquired signals using a smartphone and a spirometer, after alignment, for two breathing maneuvers performed by one subject.

FIG. 7 is a plot of an example of simultaneously-acquired data using a smartphone camera and a spirometer for one subject's experiment.

FIG. 8 is a plot of an example of the V_{T} estimation using the smartphone data calibrated via the IS for the test maneuver of a subject as well as a plot of corresponding estimation errors with respect to reference volume from spirometry.

FIG. 9 is a plot of tidal volume estimation from smartphone-acquired chest movement signal calibrated using incentive spirometer, IS, (N=12 subjects).

FIG. 10 is a plot of tidal volume estimated via linear regression of smartphone-acquired data and reference tidal volume from spirometer (N=12 subjects).

FIGS. 11A-D show example screenshots of a smartphone application prototype for tidal volume estimation using a camera and calibration with an incentive spirometer (IS).

FIG. 12 is an example of an experimental setup.

FIG. 13 is a plot of example raw volume acquired with a spirometer and the corresponding chest movement signal acquired online with the smartphone's camera and an embodiment chest movement software application (“app”).

FIG. 14 is a plot of an example of preprocessed reference volume and chest movement signals.

FIG. 15A is a flow diagram of a signal preprocessing stage.

FIG. 15B is a flow diagram of a tidal volume estimation stage.

FIG. 15C is a flow diagram of an instantaneous respiration rate (IRR) estimation stage.

FIG. 16 is a plot of an example relationship between the absolute peak-to-peak amplitude of chest movement acquired with a smartphone and a plot of reference tidal volume acquired with the spirometer for each breath phase of the maneuver performed by one subject.

FIGS. 17A-C are plots of an example V_{T} estimation using smartphone-acquired data.

FIG. 18A is a plot of linear regression results.

FIG. 18B is a Bland-Altman plot corresponding to FIG. 18A.

FIGS. 19A-C are plots of IRR estimation.

FIG. 20A is a plot of regression line parameters.

FIG. 20B is a Bland-Altman plot corresponding to FIG. 20A.

FIG. 21 is a block diagram of an example internal structure of a computer optionally within an embodiment disclosed herein.

### DETAILED DESCRIPTION

A description of example embodiments of the invention follows.

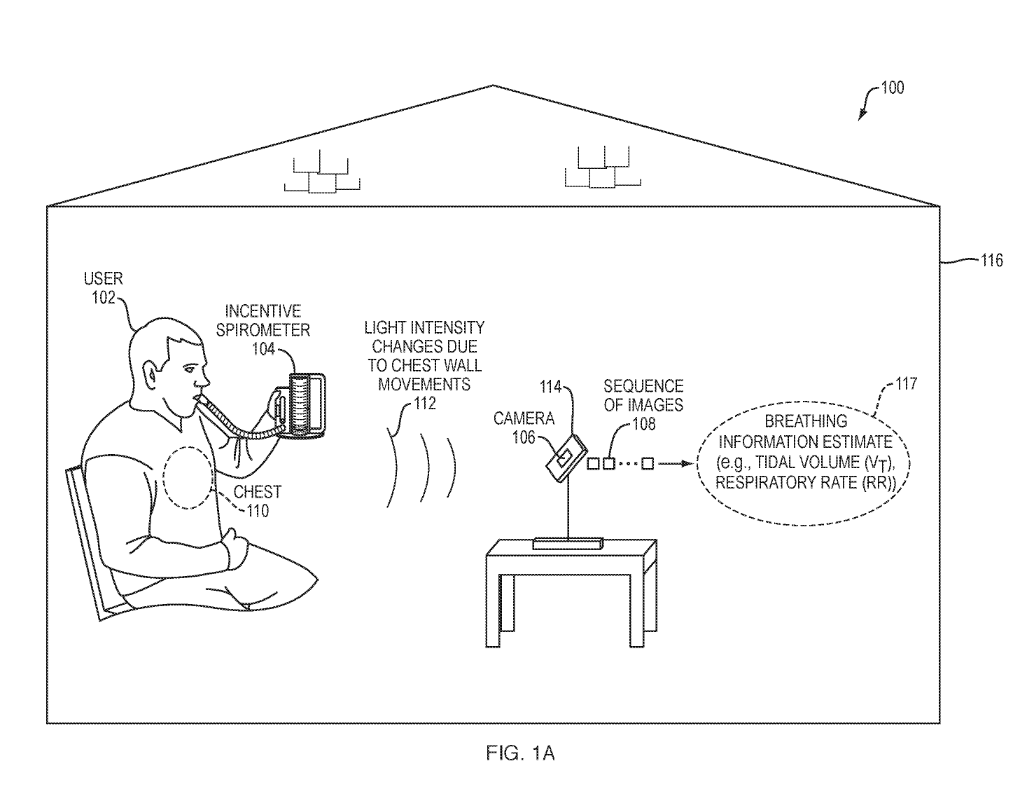

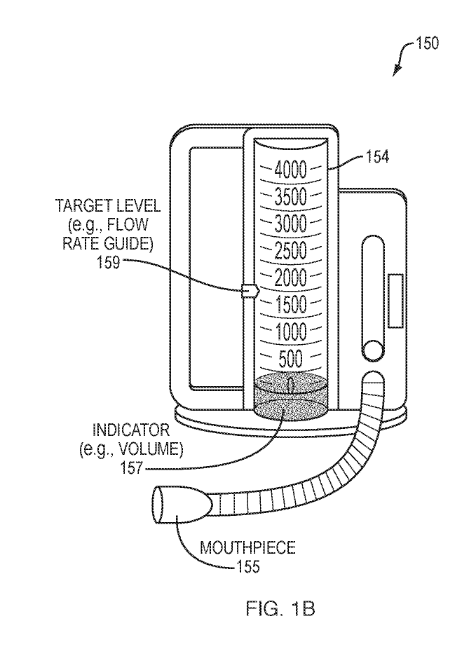

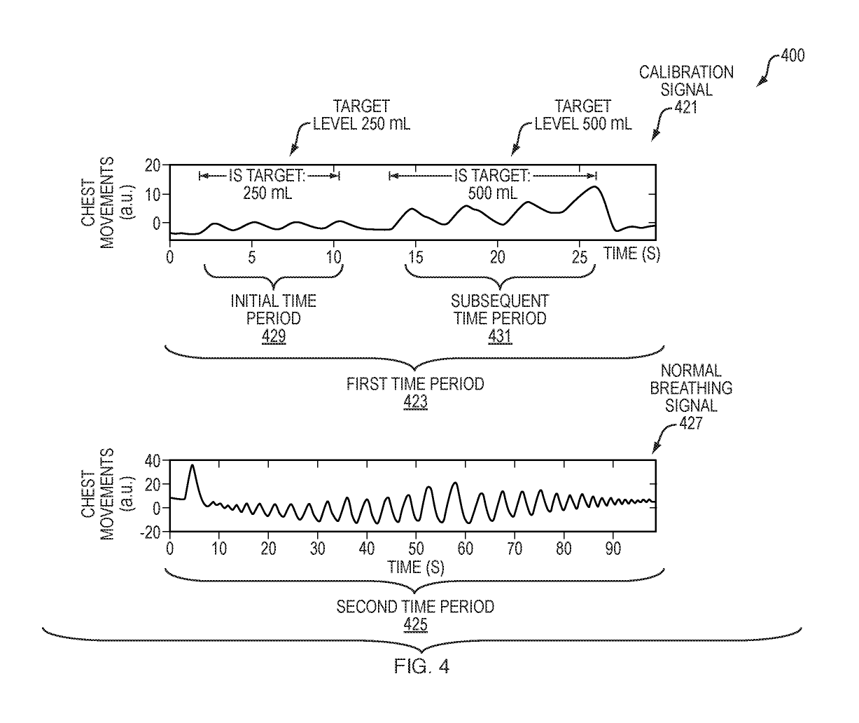

FIG. 1A is a block diagram 100 of an example application for which embodiments disclosed herein may be applied. In the block diagram 100, a user 102 is at home 116 using an incentive spirometer 104. A camera 106 may be placed in front of the user 102 at a chest level (i.e., thorax level) corresponding to the user 102′s chest 100 such that a sequence of images 108 may be recorded by the camera 106. The sequence of images 108 of the user's chest 110 may be used by a processor (not shown) to compute a calibration signal, such as the calibration signal 421 of FIG. 4, disclosed below.

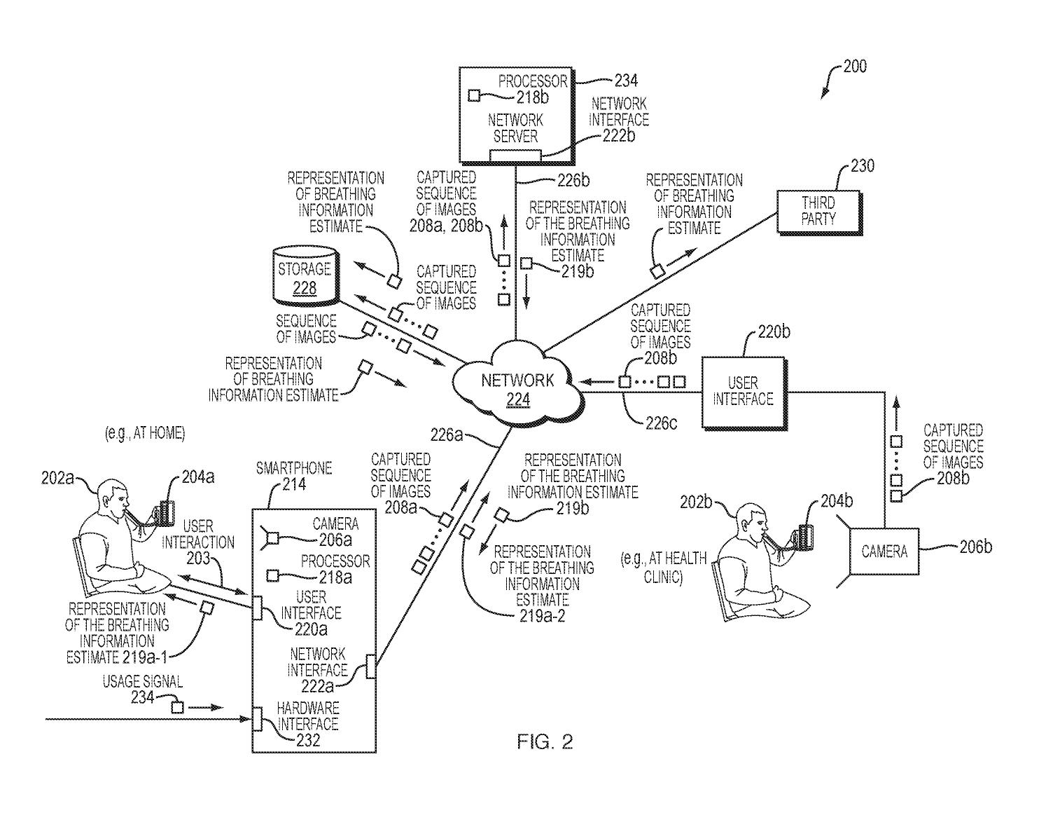

According to some embodiments, the processor (not shown) may be included in a smartphone 114 that includes the camera 106. Alternatively, according to some embodiments, the processor may be remote with respect to the camera 106, as disclosed below with reference to FIG. 2. The calibration signal may be representative of movement of the user's chest 110 as light intensity changes 112 due to chest wall movements. The movement may be captured in the sequence of images 108 and used for computing the calibration signal. The sequence of images 108 may include a first sequence of images (not shown) of the user's chest 110 and a second sequence of images (not shown) of the user's chest 110. The calibration signal may be representative of movement of the user's chest 110 during a first time period (not shown), such as the first time period 423 of FIG. 4, disclosed below. The first time period may be a time during which the user 102 is using the incentive spirometer 104.

The first sequence of images may correspond to the first time period. The processor may be configured to compute the calibration signal from the first sequence of images of the user's chest 110 to produce a calibration model (not shown). As disclosed below, the calibration model may be a linear model. The processor may be further configured to employ the calibration model to produce a breathing information estimate 117 about the user's breathing from the second sequence of images of the user's chest 110. The second sequence of images may correspond to second time period during which the user 102 is not using the incentive spirometer 104. The breathing information estimate 117 may include a representation of tidal volume, respiratory rate, or instantaneous respiratory rate, or a combination thereof, or any other suitable breathing information estimate or combination thereof.

The incentive spirometer 104 is a portable, non-invasive, low-cost monitoring device that is accessible to and easily used by the general population, such as the user 102 using the incentive spirometer 104 at home 116. As such, according to embodiments disclosed herein, the user 102 may obtain a breathing information estimate 117 with a low-cost solution and the convenience of being at home 116.

FIG. 1B is a block diagram 150 of an example embodiment of an incentive spirometer 154 that may be used by a user, such as the user 102 of FIG. 1A. A user may be referred to interchangeably herein as a person, patient, or subject. To use the incentive spirometer 154, the user may exhale (i.e., breathe out), normally, and then put the mouthpiece 155 in his or her mouth and seal lips tightly around the mouthpiece 155. The user then inhales slowly and deeply through the mouthpiece 155 to raise the indicator 157, that may be a piston, to the target level 159 (e.g., flow rate guide). The user then holds his or her breath as long as possible and then exhales, slowly, allowing the indicator 157 to fall to the bottom of the column of the incentive spirometer 154. Then the user remove's the mouthpiece 155 and rests for a few seconds before repeating again. As such, a user, such as the user 102 of FIG. 1A, may use the incentive spirometer and using the incentive spirometer, may include inhaling through the incentive spirometer 154 or exhaling through the incentive spirometer 154.

FIG. 2 is a block diagram of an example embodiment of a system 200. In the system 200, a user 202a may be using an incentive spirometer 204a at a location, such as home. It should be understood that home is one example of the location and that the location may be any suitable location. The user 202a may have a smartphone 214 for monitoring the user's breathing. The smartphone 214 may comprise a processor 218a. The processor 218a may be configured to compute a calibration signal (not shown), such as the calibration signal 421, disclosed below with reference to FIG. 4.

The calibration signal may be computed from a first sequence of images (not shown) of the user's chest to produce a calibration model (not shown). The calibration signal may be representative of movement of the user's chest during a first time period (not shown), such as the first time period 423, disclosed below with reference to FIG. 4, during which the user 202a may be using the incentive spirometer 104a. The first sequence of images may correspond to the first time period. The processor 218a may be further configured to employ the calibration model to produce a breathing information estimate (not shown), such as tidal volume, respiratory rate, instantaneous respiratory rate, or a combination thereof, about the user's breathing from a second sequence of images (not shown) of the user's chest. The second sequence of images may correspond to a second time period, such as the second time period 425 of FIG. 4, disclosed below, during which the user 202a is not using the incentive spirometer 204a.

The smartphone 214 may further include an integrated camera 206a, configurable to capture the first sequence of images and the second sequence of images. The smartphone 214 may further include a user interface 220a. The processor 218a may be further configured to determine the first time period and the second time period based on user interactions 203 with the user 202a via the user interface 220a.

The processor 218a may be further configured to output a representation of the breathing information estimate 219a-1 via the user interface 220a. The representation of the breathing information estimate 219a-1 may, for example, be displayed on a screen on the user's smartphone 214. Alternatively or in combination, the representation of the breathing information estimate 219a-1 may be output in the form of audio from the user's smartphone 214. It should be understood that the smartphone 214 may be any suitable personal device that has the integrated camera 206a and the processor 218a.

The smartphone 214 may further include a network interface 222a. The processor 218a may be further configured to output the representation of the breathing information estimate 219a-2 via the network interface 222a. The representation of the breathing information estimate 219a-2 may be output in any suitable way, such as a text message, email, or any other suitable form of data, via a communication 226a path to a network 224. The representation of the breathing information estimate 219a-2 may sent to another device (not shown), such as a device associated with a third party 230, storage location 228, or network server 234, each being communicatively coupled to the network 224. The third party 230 may be any suitable third party, such as a doctor of the user 202a.

The smartphone 214 may further include a hardware interface 232 configured to detect a usage signal 234. The usage signal 234 may represent usage of the incentive spirometer 204a. The processor 218a may be further configured to determine the first and second time periods based on detection of the usage signal 234. Detection of the usage signal 234 may include detection of start and stop indications indicated by the usage signal 234. The usage signal 234 may include start and stop indications (not shown) for cycles of use of the incentive spirometer 204a. The usage signal 234 may be triggered via a trigger component (not shown) that is communicatively coupled to the hardware interface 232. The trigger component may be any suitable device that the user 202a may use to indicate a start and end of a cycle of use of the incentive spirometer 204a. Alternatively, the processor 218a may determine the start and end of the cycle of use based on the user interactions 203.

For example, the user may indicate the start of the cycle of use via the user interface 220a in any suitable way, for example, via audio input to the user interface 220, pressing on a visual indication or button on the user interface 220a, or in any other suitable way. In a similar fashion, the end of the cycle of use may be determined based on the user interactions 203. It should be understood that the user interactions 203 may be bi-directional. For example, the processor 218a may guide the user 202a through a usage cycle of the spirometer 204a via the user interface 220a via prompts for starting and stopping the usage cycle. Corresponding information regarding a target level (i.e., an incentive level) for the usage cycle, such as the target level 159 of FIG. 1B, disclosed above, may be included in the user interactions 203.

According to some embodiments, the processor 218a may determine the start and end of the usage cycle based on processing sequences of images from the camera 206a to detect a signature of the use that may be a characteristic of, for example, an inhalation or exhalation phase of use of an incentive spirometer. As shown in FIG. 4, disclosed below, the calibration signal 421 has different pattern characteristics from those of a normal breathing signal 427 associated with normal breathing (i.e., no incentive spirometer usage). As such, sequences of images corresponding to the calibration and normal breathing signals may be understood to also have different characteristics as chest movements may be different causing different light intensity changes, such as the light intensity changes 112 disclosed above with reference to FIG. 1A.

The system 200 may include the network server 234. The network server 234 may be communicatively coupled to the network 224 via a communication path 226b. The network server 234 may include a network interface 222b. The network server 234 may be configured to receive a captured sequence of images 208a via the network interface 222b. The captured sequence of images 208a may have been captured by the camera 206a, for processing at the network server 234, that is, remotely, and may include the first sequence of images (not shown) and the second sequence of images (not shown) captured by the camera 206a, as disclosed above. The network server 234 may be configured to receive a captured sequence of images 208a via the network interface 222b. In addition, the network server 234 may be configured to receive a captured sequence of images 208b of a user 202b's chest via the network interface 222b.

The captured sequence of images 208b of the user's 202b chest may be captured by a camera 206b at another location, such as a health clinic, or other suitable location. The captured sequence of images 208b may be sent to the storage location 228 or the network server 234 via a user interface 220b communicatively coupled to the network 224 via a communication path 226c. According to some embodiments, the captured sequence of images 208a and 208b may have been stored at the storage location 228.

The network server 234 may include a processor 218b. The processor 218b may be configured to compute respective calibration signals (not shown), such as the calibration signal 421, disclosed below with reference to FIG. 4, from respective first sequences of images (not shown) of the user 202a's and the user 202b's chests to produce respective calibration models (not shown). The respective calibration signals may be representative of movement of the users' 202a and 202b chests during respective first time periods (not shown), such as the first time period 423, disclosed below with reference to FIG. 4, during which the respective users 202a and 202b may be using respective incentive spirometers 204a and 204b.

The respective first sequence of images may correspond to respective first time periods. The processor 218b may be further configured to employ the respective calibration models to produce respective breathing information estimates (not shown), such as respective tidal volume, respiratory rate, instantaneous respiratory rate, or a combination thereof, about the user 202a's breathing and user 202b's breathing from respective second sequences of images (not shown) of the respective user 202a's chest and the user 202b's chest. The respective second sequences of images may correspond to respective second time periods, such as the second time period 425 of FIG. 4, disclosed below, during which the users 202a and 202b are not using the respective incentive spirometers 204a and 204b.

The network server 234 may be further configured to output a respective representation of the breathing information estimate 219b via the network interface 222b that may be sent to the storage location 228, third party 230, smartphone 214 (e.g., for remote processing applications in which the smartphone 214 does not include the camera 206a), combination thereof, or any other suitable destination.

Similar to the smartphone 214, disclosed above, the processor 218b of the network server 234 may determine respective start and end usage cycles corresponding to use of the incentive spirometer 204a by the user 202a, and to use of the incentive spirometer 204b by the user 202b. The respective start and end usage cycles may be determined based on the respective captured sequence of images 208a and 208b, captured by the camera 206a and 206b, respectively, to detect a respective signature of the use that may be a characteristic of, for example, an inhalation or exhalation phase of use of an incentive spirometer.

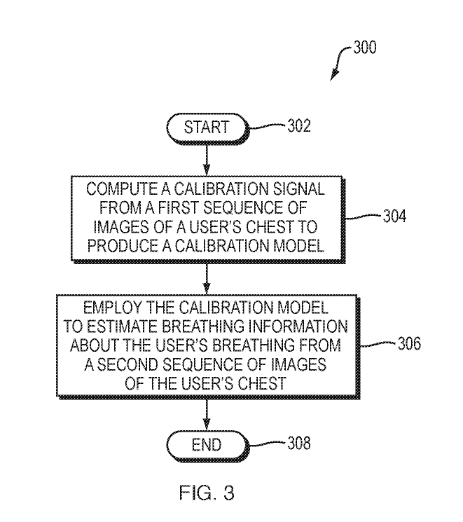

FIG. 3 is a flow diagram 300 of an example embodiment of a method. The method may start (302) and compute a calibration signal from a first sequence of images of a user's chest to produce a calibration model, the calibration signal representative of movement of the user's chest during a first time period during which the user is using an incentive spirometer, the first sequence of images corresponding to the first time period (304). The method may employ the calibration model to produce a breathing information estimate about the user's breathing from a second sequence of images of the user's chest corresponding to a second time period during which the user is not using the incentive spirometer (306) and the method may end (308) in the example embodiment.

FIG. 4 is a block diagram 400 of plots of signals representative of a user's chest movements, such as chest movements of the user 102 and the users 202a-b, disclosed above with reference to FIG. 1A and FIG. 2. As disclosed above, a calibration signal may be computed from a first sequence of images of a user's chest to produce a calibration model. The calibration signal 421 may be an example of such a calibration signal that is computed by a processor. A first time period 423 is shown and may include at least two time periods, an initial time period 429 and a subsequent time period 431, during which a user is using an incentive spirometer. The user may achieve a different target level on the incentive spirometer during respective periods of the at least two time periods, for example 250 mL in the initial time period and 500 mL in the subsequent time period. It should be understood that 250 mL and 500 mL are example target levels and that any suitable target level may be used.

The first sequence of images, disclosed above, may include at least two sequences of images corresponding to the at least two time periods, such as the initial time period 429 and the subsequent time period 431. As such, the calibration signal 421 may be further representative of movement of the user's chest during respective periods of the at least two time periods, such as the initial time period 429 and the subsequent time period 431.

In FIG. 4, a plot of a normal signal 427 is shown. The normal signal 427 may be computed from a second sequence of images of the user's chest that correspond to a second time period 425 during which the user is not using the incentive spirometer. It should be understood that the plots shown in FIG. 4 are for illustrative purposes only.

It should be understood that any of the following embodiments may be used with any of the embodiments disclosed above.

An embodiment of a smartphone-based tidal volume (V_{T}) estimator was developed, where an Android® application provides a chest movement signal whose peak-to-peak amplitude is highly correlated with reference V_{T} measured by a spirometer. It was found that a Normalized Root Mean Squared Error (NRMSE) of 14.998±5.171% (mean±SD) when the smartphone measures were calibrated using spirometer data. However, the availability of a spirometer device for calibration is not realistic outside clinical or research environments. In order to be used by the general population on a daily basis, a simple calibration procedure not relying on specialized devices may be useful.

Embodiments disclosed herein may take advantage of the linear correlation between smartphone measurements and V_{T} to obtain a calibration model using information computed while the subject breathes through a commercially-available incentive spirometer (IS). Experiments were performed on twelve (N=12) healthy subjects. It was found that the calibration procedure using an IS resulted in a fixed bias of −0.051 L and a RMSE of 0.189±0.074 L corresponding to 18.559±6.579% when normalized. Although it has a small underestimation and slightly increased error, the calibration procedure using an IS has the advantages of being simple, fast, and affordable. Results of testing embodiments disclosed herein support the feasibility of developing a portable smartphone-based breathing status monitor that provides information about breathing depth, in addition to the more commonly estimated respiratory rate, on a daily basis.

Tidal volume (V_{T}) provides information about the breathing depth and is defined as the volume of air moved with each breath. Normal average V_{T} is approximately 0.5 L but this volume varies as the mechanism of respiratory control adjusts both it and respiratory rate (RR) in response to different activities, for example exercise or sleep, to meet the body's requirements (Koeppen, B. M.; Stanton, B. A. Berne & Levy Physiology, Updated Edition; Elsevier Health Sciences, 2009). Tidal volume is important information to measure during mechanical ventilation to ensure sufficient ventilation without trauma to the lungs. Moreover, for patients with chronic obstructive pulmonary diseases, having the luxury to estimate their tidal volume at homes could be beneficial. For example, upon asthma attack, having not only the respiratory rate but also the tidal volume of the patient would give a physician better quantification of the severity of the asthma attack.

Several clinical and research methods currently exist to estimate V_{T} including spirometry, impedance pneumography, inductance plethysmography, photoplethysmography, Doppler radar, computed tomography, phonospirometry, and electrocardiography (Ashutosh, K.; Gilbert, R.; Auchincloss, J. H.; Erlebacher, J.; Peppi, D. Impedance pneumograph and magnetometer methods for monitoring tidal volume. J Appl Physiol 1974, 37, 964-966; Grossman, P.; Spoerle, M.; Wilhelm, F. H. Reliability of respiratory tidal volume estimation by means of ambulatory inductive plethysmography. Biomed. Sci. Instrum. 2006, 42, 193-198; Johansson, A.; Öberg, P. P. A. Estimation of respiratory volumes from the photoplethysmographic signal. Part I: experimental results. Med. Biol. Eng. Comput. 1999, 37, 42-47; Lee, Y. S.; Pathirana, P. N.; Steinfort, C. L.; Caelli, T. Monitoring and Analysis of Respiratory Patterns Using Microwave Doppler Radar. IEEE J. Transl. Eng. Health Med. 2014, 2, 1-12; Li, G.; Arora, N. C.; Xie, H.; Ning, H.; Lu, W.; Low, D.; Citrin, D.; Kaushal, A.; Zach, L.; Camphausen, K.; Miller, R. W. Quantitative prediction of respiratory tidal volume based on the external torso volume change: a potential volumetric surrogate. Phys. Med. Biol. 2009, 54, 1963-1978; Miller, M. R.; Hankinson, J.; Brusasco, V.; Burgos, F.; Casaburi, R.; Coates, A.; Crapo, R.; Enright, P.; van der Grinten, C. P. M.; Gustafsson, P.; others Standardisation of spirometry. Eur. Respir. J. 2005, 26, 319-338; Que, C.-L.; Kolmaga, C.; Durand, L.-G.; Kelly, S. M.; Macklem, P. T. Phonospirometry for noninvasive measurement of ventilation: methodology and preliminary results. J. Appl. Physiol. Bethesda Md 1985 2002, 93, 1515-1526; Sayadi, O.; Weiss, E. H.; Merchant, F. M.; Puppala, D.; Armoundas, A. A. An Optimized Method for Estimating the Tidal Volume from Electrocardiographic Signals: Implications for Estimating Minute Ventilation. Am. J. Physiol.—Heart Circ. Physiol. 2014, 307, H426-H436; Semmes, B. J.; Tobin, M. J.; Snyder, J. V.; Grenvik, A. Subjective and objective measurement of tidal volume in critically ill patients. Chest 1985, 87, 577-579). However, these devices have been largely designed for clinical or research centers and hence they are not applicable for everyday use for home monitoring due to the complexity of the devices, their high cost, their need for skilled operators, and in some cases their limited portability.

An interesting approach to overcome some of the abovementioned limitations is to use general purpose video cameras to optically monitor breathing. Although most efforts in this area have focused on estimation of the RR (Bartula, M.; Tigges, T.; Muehlsteff, J. Camera-based system for contactless monitoring of respiration. In 2013 35th Annual International Conference of the IEEE Engineering in Medicine and Biology Society (EMBC); 2013; pp. 2672-2675; Poh, M.-Z.; McDuff, D. J.; Picard, R. W. Advancements in Noncontact, Multiparameter Physiological Measurements Using a Webcam. IEEE Trans. Biomed. Eng. 2011, 58, 7-11; Tarassenko, L.; Villarroel, M.; Guazzi, A.; Jorge, J.; Clifton, D. A.; Pugh, C. Non-contact video-based vital sign monitoring using ambient light and auto-regressive models. Physiol. Meas. 2014, 35, 807; Wu, H.-Y.; Rubinstein, M.; Shih, E.; Guttag, J.; Durand, F.; Freeman, W. Eulerian Video Magnification for Revealing Subtle Changes in the World. ACM Trans Graph 2012, 31, 65:1-65:8; Zhao, F.; Li, M.; Qian, Y.; Tsien, J. Z. Remote Measurements of Heart and Respiration Rates for Telemedicine. PLoS ONE 2013, 8, e71384; Nam, Y.; Kong, Y.; Reyes, B.; Reljin, N.; Chon, K. H. Monitoring of Heart and Respiratory Rates using Dual Cameras on a Smartphone. PLoS ONE 2016, In Press), there have also been studies to estimate V_{T} (Ferrigno, G.; Carnevali, P.; Aliverti, A.; Molteni, F.; Beulcke, G.; Pedotti, A. Three-dimensional optical analysis of chest wall motion. J. Appl. Physiol. Bethesda Md 1985 1994, 77, 1224-1231; Cala, S. J.; Kenyon, C. M.; Ferrigno, G.; Carnevali, P.; Aliverti, A.; Pedotti, A.; Macklem, P. T.; Rochester, D. F. Chest wall and lung volume estimation by optical reflectance motion analysis. J. Appl. Physiol. 1996, 81, 2680-2689; Shao, D.; Yang, Y.; Liu, C.; Tsow, F.; Yu, H.; Tao, N. Noncontact Monitoring Breathing Pattern, Exhalation Flow Rate and Pulse Transit Time. IEEE Trans. Biomed. Eng. 2014, 61, 2760-2767; Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press.) Recently, a volume conservation hypothesis was proposed by establishing a one-to-one linear relationship between changes of the external torso volume and V_{T} corresponding to internal lung air content (Li, G.; Arora, N. C.; Xie, H.; Ning, H.; Lu, W.; Low, D.; Citrin, D.; Kaushal, A.; Zach, L.; Camphausen, K.; Miller, R. W. Quantitative prediction of respiratory tidal volume based on the external torso volume change: a potential volumetric surrogate. Phys. Med. Biol. 2009, 54, 1963-1978). Previous findings also indicated that accurate V_{T} estimation results by tracking markers placed on the chest wall surface via an optical reflectance system (Ferrigno, G.; Carnevali, P.; Aliverti, A.; Molteni, F.; Beulcke, G.; Pedotti, A. Three-dimensional optical analysis of chest wall motion. J. Appl. Physiol. Bethesda Md 1985 1994, 77, 1224-1231; Cala, S. J.; Kenyon, C. M.; Ferrigno, G.; Carnevali, P.; Aliverti, A.; Pedotti, A.; Macklem, P. T.; Rochester, D. F. Chest wall and lung volume estimation by optical reflectance motion analysis. J. Appl. Physiol. 1996, 81, 2680-2689). Although promising, these approaches are difficult to apply to the general population outside research setting do the reasons listed above.

More recently, a good correlation (r²=0.81) between shoulder displacements obtained by processing webcam video recordings and exhaled breath volume measured with a commercial metabolic analysis device was obtained (Shao, D.; Yang, Y.; Liu, C.; Tsow, F.; Yu, H.; Tao, N. Noncontact Monitoring Breathing Pattern, Exhalation Flow Rate and Pulse Transit Time. IEEE Trans. Biomed. Eng. 2014, 61, 2760-2767). Besides the promising results, analysis in terms of V_{T} estimation was limited to the correlation between the video amplitudes and reference volumes. In addition, the implementation was done in a personal computer and with the aid of an external digital camera.

Smartphones' fast microprocessors, multiple sensors, large data storage, software flexibility, and media capabilities are attractive for developing monitoring systems that can potentially be used by the general public. They have, accordingly, been found to be accurate in a diversity of vital sign monitoring applications (Scully, C.; Lee, J.; Meyer, J.; Gorbach, A. M.; Granquist-Fraser, D.; Mendelson, Y.; Chon, K. H. Physiological parameter monitoring from optical recordings with a mobile phone. Biomed. Eng. IEEE Trans. On 2012, 59, 303-306; Lee, J.; Reyes, B. A.; McManus, D. D.; Mathias, O.; Chon, K. H. Atrial Fibrillation Detection Using an iPhone 4S. IEEE Trans. Biomed. Eng. 2013, 60, 203-206; Nam, Y.; Lee, J.; Chon, K. H. Respiratory Rate Estimation from the Built-in Cameras of Smartphones and Tablets. Ann. Biomed. Eng. 2013, 42, 885-898; Reljin, N.; Reyes, B. A.; Chon, K. H. Tidal Volume Estimation Using the Blanket Fractal Dimension of the Tracheal Sounds Acquired by Smartphone. Sensors 2015, 15, 9773-9790). An approach was investigated using dual cameras consisting of contact and noncontact video monitoring directly implemented on a smartphone for estimation of heart rate (HR) and the mean RR, respectively (Nam, Y.; Kong, Y.; Reyes, B.; Reljin, N.; Chon, K. H. Monitoring of Heart and Respiratory Rates using Dual Cameras on a Smartphone. PLoS ONE 2016, In Press). In that study, noncontact video monitoring of the chest area provided waveforms whose amplitudes were concordant with either the increase or decrease in the depth of breathing. In a subsequent study, use of the non-contact approach for the task of V_{T} estimation and tracking of RR at each time instant was analyzed (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press). It was found that the peak-to-peak amplitude of the smartphone-acquired chest movement signal was highly correlated with the V_{T} from the spirometer, which was regarded as the reference (r²=0.951±0.042, mean±SD). It was found that when calibrated on an individual basis, the root-mean-squared error was 0.182±0.107 L, which is equivalent to 14.998±5.171% when normalized.

According to embodiments disclosed herein, a calibration method may use a volume-oriented incentive spirometer (IS) for the task of V_{T} estimation from the smartphone-acquired chest movement signal. To this end, the V_{T} estimates were computed after calibration from data computed while breathing through an IS, and compared to simultaneously-acquired volume from a spirometer as reference. The performance of the V_{T} estimation from the calibration method via IS was also compared to the best estimation that could be obtained via linear regression between the reference volume and smartphone data. The smartphone application, according to embodiments disclosed herein, was implemented in a commercially-available Android® smartphone and its screens used for V_{T} estimation are described herein.

2. Material and Methods

2.1. Subjects

Twelve (N=12) healthy and non-smoker volunteers (eleven males) aged 27.7±9.5 years (mean±standard deviation), weight 71.6±7.8 kg, and height 174.5±6.0 cm, were recruited for a study. Individuals with previous pneumothorax, those with chronic respiratory illnesses such as asthma, and anyone who had symptoms of the common cold or an upper respiratory infection were excluded from this study. Each volunteer consented to be a subject and signed the study protocol approved by the Institutional Review Board of the University of Connecticut (UConn, Storrs, Conn., USA).

2.2. Signal acquisition

Equipment

The method for recording the chest movement signal was implemented in an HTC One M8 smartphone (HTC Corporation, New Taipei City, Taiwan) running the Android® v4.4.2 operating system. The frontal camera of this smartphone was used, which had a 5 MP, backside-illumination sensor with wide angle lens and 1080p full HD video recording capabilities at 30 frames-per-second. The implemented application processed the video data in real time to obtain the chest movement signal for estimation of V_{T}. Collected data were saved into a text file for offline analysis of results in Matlab® (R2012a, The Mathworks, Natick, Mass., USA).

To test the smartphone-based V_{T} estimates, a reference volume signal was collected with a spirometer system consisting of a respiration flow head connected to a differential pressure transducer for measuring the airflow signal (MLT1000L, FE141 Spirometer, ADlnstruments, Dunedin, New Zealand). The integral of the airflow over time was computed to generate the volume signal. Both the airflow and volume signals were sampled at 1 kHz using a 16-bit A/D converter (PowerLab/4SP, ADlnstruments). Prior to recording, the spirometer system was calibrated using a 3.0 L calibration syringe (Hans Rudolph, Inc., Shawnee, Kans., USA). Each volunteer was provided with a new breathing apparatus set consisting of a disposable filter, mouthpiece, and nose clip (MLA304, MLA1026, MLA1008, ADlnstruments). For calibration of the smartphone-based V_{T} estimates, a new volumetric incentive spirometer (IS) was provided to each volunteer (Airlife™, Carefusion, Yorba Linda, Calif., USA).

Breathing maneuvers

Each experiment consisted of two phases with the corresponding maneuvers as follows:
I. Calibration Maneuver

Volunteers were asked to breathe four times through the IS, inhaling to a first target of 250 mL, then hold their breath for 2 seconds, and finally breathe four times through the IS to a second inhalation target of 500 mL.
II. Test Breathing Maneuver

Volunteers were asked to hold their breath for 2 seconds, take a deep breath, and then breathe through the spirometer system to different inhalation volume levels ranging from around 200 mL to 2.5 L; first increasing their V_{T} with each inhalation for around one minute, and finally decreasing their V_{T} with each inhalation for another minute. Subjects breathed at their own pace, i.e., a metronome to control their respiratory frequency was not used. Reference volume was recorded for this maneuver using spirometry.

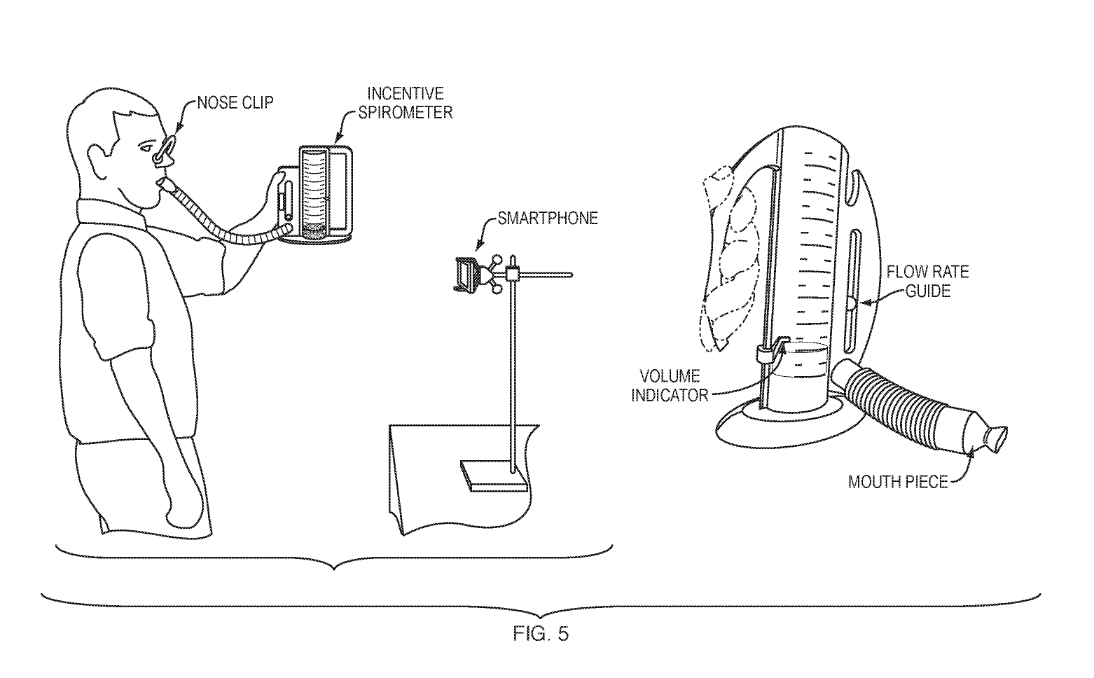

Data from the calibration maneuver was used to compute the calibration model for the smartphone-based V_{T} estimates. As seen in FIG. 5, disclosed below, the IS used has increments of 250 mL, a volume indicator, and a flow rate guide. Volunteers were asked to hold the IS in its upright position and then breathe through the mouthpiece of the IS so that at each inspiration the top of the volume indicator lined up with the corresponding target mark, while the flow rate indicator was kept in between the two arrow guides to maintain an adequate inspiration speed as indicated in the manufacturer's manual.

While the volunteers performed the calibration maneuver, the chest movement signal was recorded using the smartphone placed in front of the subject at approximately 60 cm in a 3-pronged clamp at their thoracic level. It is worth mentioning that the volume signal from the spirometer was not recorded during the calibration maneuver as the volunteers were breathing through the IS mouthpiece. Hence, the exact volume inspired at each breathing phase of the calibration maneuver was not collected, but fixed at the predefined target levels. Before starting the calibration maneuver, the volunteers learned how to use the IS and were allowed to practice and familiarize themselves with the maneuver. The smartphone application according to embodiments disclosed herein allows a remote Start/Stop recording option via a generic Bluetooth® camera shutter (I Shutter, Shanghai, China).

The second (test) maneuver provided a wide range of V_{T} to test the computed calibration model. Simultaneous recording of the smartphone-based chest movement signal and spirometer-acquired reference volume was performed. The chest movement signal was recorded in the same manner as it had been for the calibration maneuver. Visual feedback was provided to the volunteers by displaying the reference volume on a 40″ monitor placed in front of them, where visual marks were used to indicate the tidal volume's range of interest.

Both maneuvers were recorded in a regular dry lab using ambient light from ceiling fluorescent lamps. During both maneuvers, subjects were asked to stand still and not to change position in between maneuvers. Nose clips were used to clamp the nostrils during both maneuvers. A concern that arises when employing a non-contact optical approach for breathing monitoring is the ability of the system to capture the breathing-related movements when the subjects are wearing different colors and patterns, as this approach looks for changes in the light intensity due to the modification of the path length caused by breathing displacements of the chest wall. Hence, during the experiments, volunteers had the freedom to wear different colored and patterned clothes like plain or stripes, and were only asked not to wear loose clothes. As with other breathing monitoring techniques, e.g. inductance plethysmography, the quality of the signal and ultimately the performance of the monitoring system could degrade if clothes are too loose to see chest movements.

FIG. 5 is a block diagram of an acquisition setup for a subject breathing through an incentive spirometer. Left: Experimental setup to record the chest movements using the smartphone camera while volunteers breathe through an incentive spirometer (IS) for calibration. Right: Detailed view of the IS while the subject is inspiring to reach a volume target. Subjects were asked to inspire so that the top of the piston lined up with the desired blue mark and at a rate that kept the indictor between the two blue guide arrows.

2.3. Smartphone Method for Recording Chest Movements.

The chest movement signal I (t) was computed in real time in the smartphone app by averaging the intensity within a rectangular region of interest (ROI) of the red, green and blue (RGB) channels at each time instant t, according to

$$I ( t ) = ( \frac{1}{3 D} ) ( {\sum }_{\{ m , n \} ∈ ROI} {i}_{R} ( m , n , t ) + {\sum }_{\{ m , n \} ∈ ROI} {i}_{G} ( m , n , t ) + {\sum }_{\{ m , n \} ∈ ROI} {i}_{B} ( m , n , t ) ) \qquad ( 1 )$$

where iₓ(m,n,t) is the intensity value of the pixel at the m-th row and n-th column of the RGB channel within the ROI containing a total of D pixels. The camera resolution was set to 320×240 pixels, and the ROI of 49×90 pixels was focused on the thoracic area of the volunteer. The sampling rate fluctuated around 25 frames-per-second during the real time monitoring. Hence, after stopping the recording, the recorded signal was cubic splined to obtain a uniform sampling rate of 25 Hz. Finally, a bandpass filter was applied to the chest movement signal between 0.01 to 2 Hz using a 50^{th} order finite impulse response (FIR) filter, designed with a Hamming window, to minimize the high frequency components not related to the breathing maneuvers and the trend in the signal. Both the cubic spline and bandpass filtering were performed in the smartphone app. The conditioned signals of the maneuvers were saved in a text file for further analysis in a personal computer.

2.4. Data Preprocessing

The reference volume signal recorded during the second phase of the experiment (the test maneuver) was analyzed offline in Matlab®. First, it was down-sampled to 25 Hz to achieve the same sampling frequency as the corresponding chest movement signal, and then bandpass filtered using a 4th-order Butterworth bandpass between 0.01 to 2 Hz applied in a forward and backward scheme to produce zero-phase distortion and minimize the start and end transients.

Due to differences in the starting times and delays between the smartphone and spirometer systems, simultaneously recorded signals were aligned using the initial breath holding and deep inspiration portion of the data and also by using the cross-correlation function, where 20 seconds in the central portion of the maneuver were extracted from each recording to compute the cross-correlation sequence and find the sample lag with the maximum cross-correlation value indicating the required samples to be shifted.

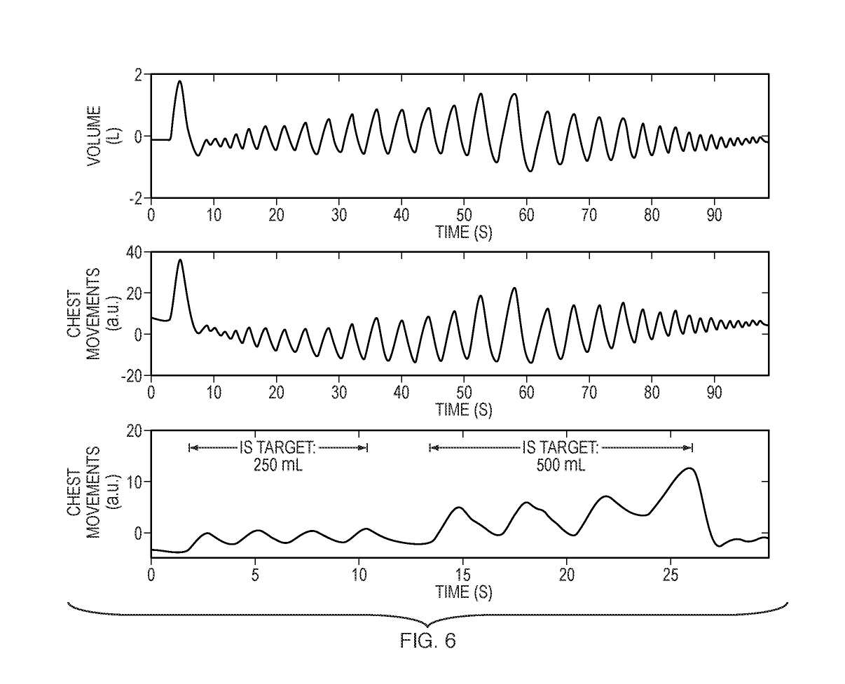

FIG. 6 shows plots of examples of acquired signals using a smartphone and a spirometer, after alignment, for two breathing maneuvers performed by one subject. Top: Reference volume from spirometer for the test maneuver. Middle: Corresponding chest movement signal for the test maneuver recorded via the smartphone camera app. These two signals from test maneuver were aligned due to different starting times. Bottom: Chest movement signal recorded during calibration maneuver while the subject was breathing though the incentive spirometer; four inspirations at 250 mL target, and four inspirations at 500 mL target. Inspirations/expirations correspond to positive/negative deflections in the signals.

2.5. Calibration Using Incentive Spirometer

The inspiratory segments of the calibration maneuver using IS were identified from the chest movement signal. Information from the expiratory segments of the calibration maneuver was not used, as the volume indicator of the IS returns to its original position mainly due to gravity and not by the expiratory effort of the volunteer. Then, the peak-to-peak amplitude of this signal was computed at each inspiration and matched with the corresponding target volume from the IS. This resulted in two data sets: 1) four data points with ordinate values V_{IS,1} at 250 mL, and 2) four data points with ordinate values V_{IS,2} at 500 mL, with each point having an abscissa Δx equal to the peak-to-peak amplitude of the chest movement signal for that corresponding inspiratory phase.

Next, the median peak-to-peak amplitude of each set was computed so that the information from the IS maneuver was condensed into two data points, A and B, as follows:

A=( [symbol] ,V_{IS,1})=( [symbol] ,250 mL)

B=( [symbol] ,V_{IS,2})=( [symbol] ,500 mL) (2)

where [symbol] and [symbol] are the median values of the peak-to-peak amplitudes of the chest movement signal for the inspirations at 250 mL target (V_{IS,1}) and at the 500 mL target (V_{IS,2}), respectively. Finally, the calibration curve to map the peak-to-peak amplitudes to tidal volume estimates was obtained using the linear equation given the locations of points A and B:

$${V}_{Tsmartphone} = ( \frac{{V}_{IS , 2} - {V}_{IS , 1}}{-} ) ( Δ x - ) + {V}_{IS , 1} \qquad ( 3 )$$

which in turn can be written as

$${V}_{Tsmartphone} = ( \frac{250}{-} ) Δ x + 250 ( 1 + \frac{-}{} ) \qquad ( 4 )$$

where V_{Tsmartphone} denotes the tidal volume estimate given the peak-to-peak amplitude of the smartphone-acquired chest movement signal and the data from calibration using IS.

2.6. Tidal Volume Estimation Using Smartphone

After the calibration linear model obtained from the calibration maneuver was applied, the V_{T} smartphone estimates were tested, using the tidal volumes obtained from the spirometer as reference. To this end, the maxima and minima of the reference volumes were found and the V_{Tspirometer} were computed as the absolute amplitude difference between two consecutive extrema. The corresponding peak-to-peak amplitudes Δx were found in the smartphone-acquired chest movement signals. Finally, the linear model obtained from the IS, given by Eq. 4, was applied to each value Δx of the maneuver to obtain the corresponding smartphone-based V_{T} estimate.

The performance of the estimation was measured on the test data in terms of the root-mean-squared error RMSE, given by

$$RMSE = \sqrt{\frac{{\sum }_{i = 1}^{M} {( {V}_{{T}_{spirometer}} ( i ) - {V}_{{T}_{smartphone}} ( i ) )}^{2}}{M}} \qquad ( 5 )$$

and its normalized version NRMSE with respect to the mean tidal volume of the maneuver, given by

$$NRMSE = \frac{RMSE}{mean ( {V}_{{T}_{spirometer}} )} \times 100 \% \qquad ( 6 )$$

where V_{T}_{ₛₚᵢᵣₒₘₑₜₑᵣ} indicates the reference tidal volume measured by the spirometer, V_{T}_{ₛₘₐᵣₜₚₕₒₙₑ} the tidal volume estimated from smartphone-acquired chest movements after calibration with the IS model, and M is the number of breath-phases of the analyzed breathing maneuver.

In addition, these V_{T} estimates obtained from calibration via IS data were compared to those V_{T} obtained when applying a linear regression to the absolute peak-to-peak amplitude of the chest movement signal and the simultaneously-recorded reference V_{T} from the spirometer, to see how much the estimates from IS calibration deviate from those obtained with the best estimation model in the least-squares sense.

3. Results

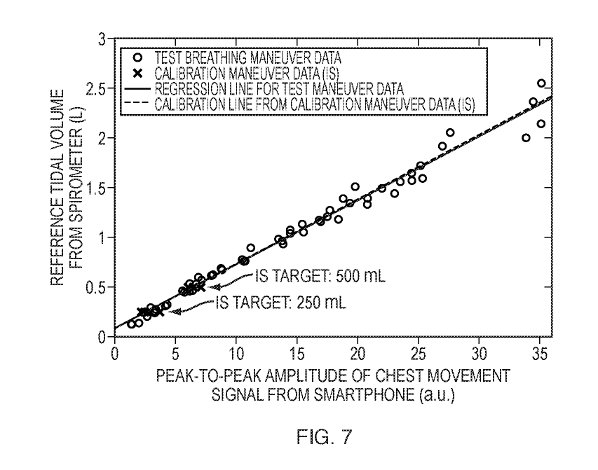

Reference tidal volumes from the spirometer distributed from a minimum of 0.190±0.116 L (mean±SD), to a maximum of 2.607±0.400 L, with an average of 1.024±0.159 L for the maneuvers performed by all volunteers (N=12). A strong linear correlation between the peak-to-peak amplitude of the chest movement signal from the smartphone's camera and the reference V_{T} from the spirometer was found (r²=0.945±0.037). An example of this relationship for the breathing maneuver of one subject is shown in FIG. 7. The distribution of r² values was not normal, as tested using a one-sample Kolmogorov-Smirnov test (p=0.017). The median r² was found to be higher than 0.9 as tested by a one-sample Wilcoxon signed rank test (p=0.002). The RMSE and NRMSE errors obtained when mapping the peak-to-peak amplitude of the chest movement signal to V_{T} quantities using linear regression is shown in Table 1.

To calibrate the peak-to-peak amplitude of the chest movement signal from the smartphone, two data points were extracted from the calibration maneuver using IS and a linear model was computed from these points to map the smartphone quantities to tidal volumes. An example of the data extracted from the calibration maneuver using IS and the corresponding calibration model are shown in FIG. 7 together with the testing data from simultaneously-recorded V_{T} from the spirometer and peak-to-peak amplitude of the chest movement signal.

FIG. 7 shows an example of simultaneously-acquired data using a smartphone camera and a spirometer for one subject's experiment. The solid gray line is the regression line for the test maneuver. Red crosses indicate the data collected during the test maneuver while the subject was breathing at 250 mL and 500 mL targets through the incentive spirometer (IS). The calibration model computed from the IS data is indicated by the red dashed line.

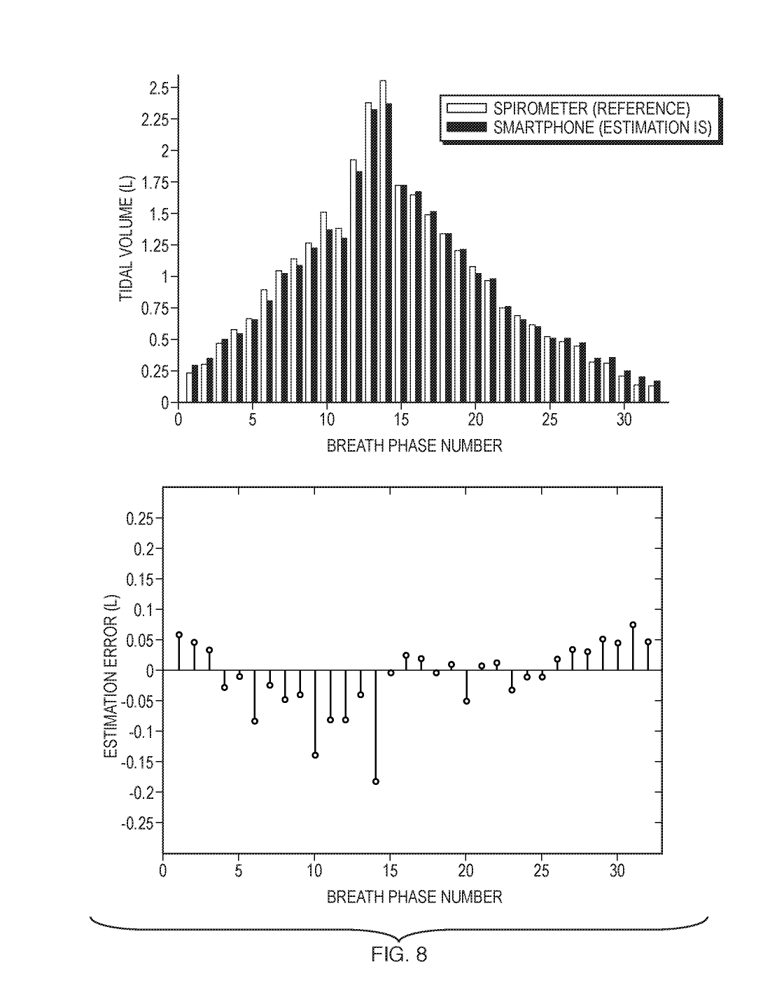

FIG. 8 shows an example of the V_{T} estimation using the smartphone data calibrated via the IS according to embodiments disclosed herein for the test maneuver of a subject as well as the corresponding estimation errors with respect to reference volume from spirometry. Table 1 shows the performance indices obtained for all subject for the V_{T} estimates from the smartphone when the calibration method via IS was used. FIG. 8 shows example plots of tidal volume estimation using the smartphone-acquired chest movement signal calibrated with an incentive spirometer (IS) for the test maneuver performed by one volunteer. For visualization purposes, only data from inspiratory phases are displayed. Top: Side-to-side tidal volumes. Bottom: Corresponding estimation errors of smartphone-system with respect to spirometry.

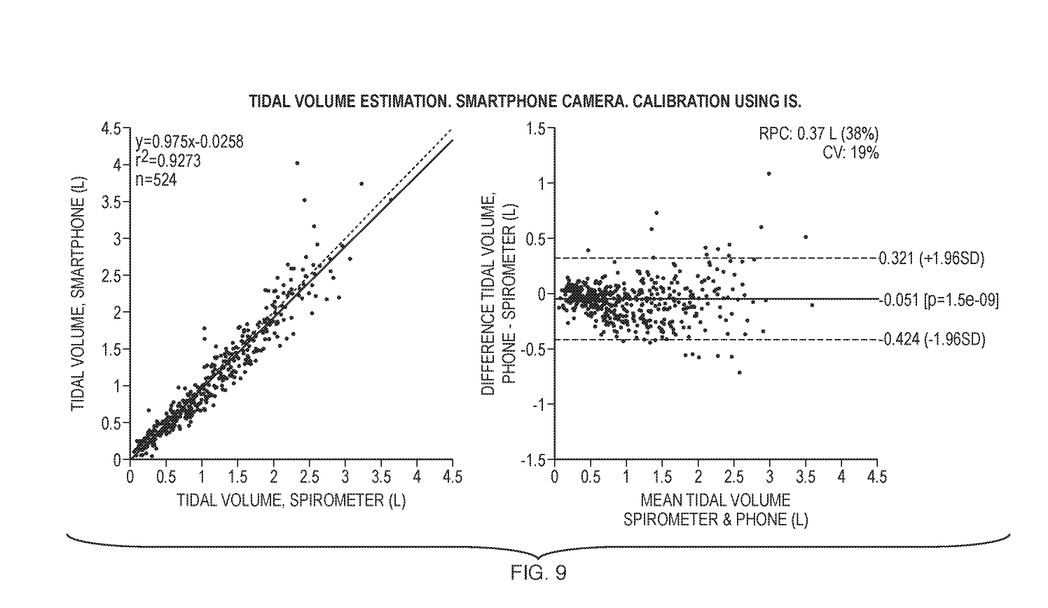

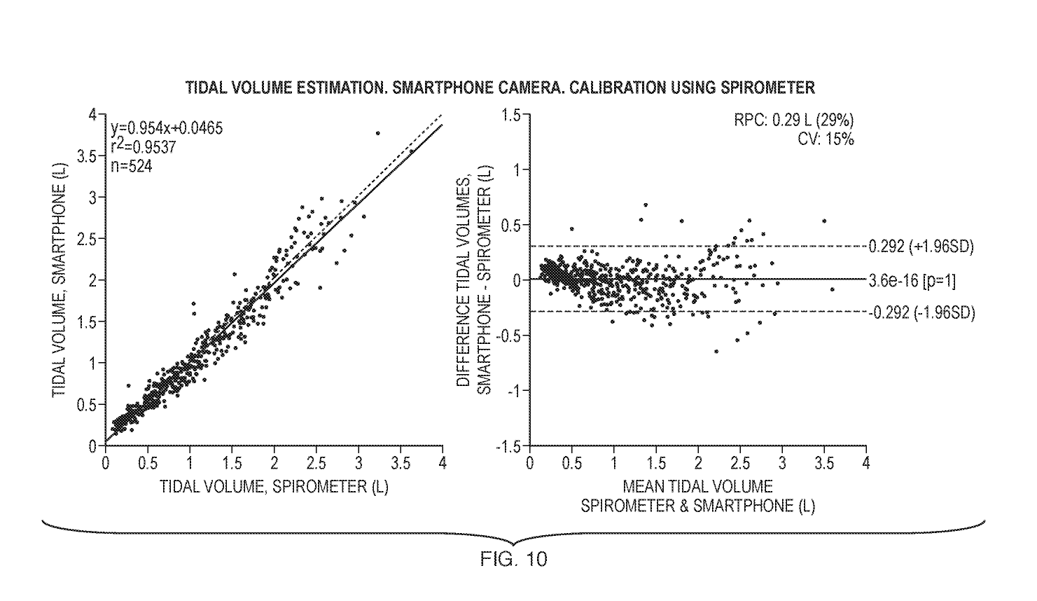

It was found that, when calibrated using the IS data, the smartphone-based V_{T} estimation produced a statistically-significant bias of −0.051 liters, and 95% limits of agreement of −0.424 and 0.321 liters, as shown in the corresponding Bland-Altman plot of FIG. 9. In contrast, when the peak-to-peak amplitudes were mapped to volumes using the linear regression of the simultaneously-acquired spirometer data, no statistically-significant bias was found, and the 95% limits of agreement were ±0.292 liters as shown in FIG. 10.

In another study using spirometer data for calibration, the RMSE and NRMSE values of the smartphone-based V_{T} estimates were found to be 0.182±0.107 L and 14.998±5.171%, respectively (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press). Those RMSE and NRMSE values did not distribute normally, as tested by a one-sample Kolmogorov-Smirnov test (p=0.008 and p=0.017, respectively). When comparing those prior results to those obtained as disclosed herein, using the best model from the regression of spirometer and smartphone data, no statistically-significant differences (p=0.961) were found. Finally, the estimation errors obtained from the calibration via IS were compared to those from the linear regression by means of a paired-sample t-test and statistically-significant increases in the mean value of the RMSE (p=0.007) and NRMSE (p=0.004) using IS were found.

FIG. 9 shows tidal volume estimation from smartphone-acquired chest movement signal calibrated using incentive spirometer, IS, (N=12 subjects). Left: Regression curve. Grey dashed line indicates the identity line and the solid black the regression line. Right: Bland-Altman plot. Solid black line indicates the bias and dashed green lines indicate the 95% limits of agreement.

FIG. 10 shows tidal volume estimated via linear regression of smartphone-acquired data and reference tidal volume from spirometer (N=12 subjects). Left: Regression curve. Grey dashed line indicates the identity line and the solid black the regression line. Right: Bland-Altman plot. Solid black line indicates the bias and dashed green lines indicate the 95% limits of agreement.

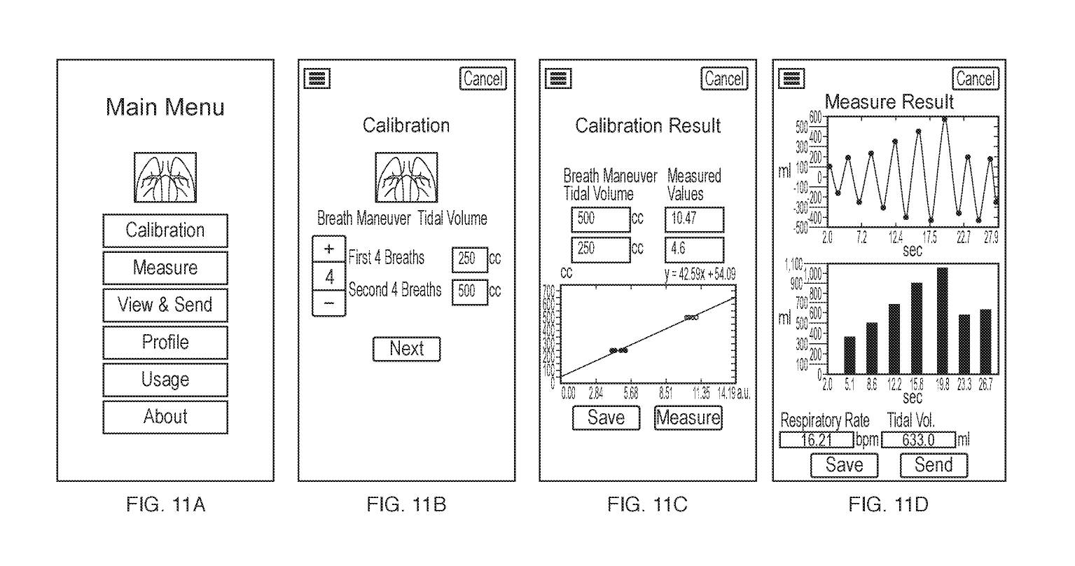

Four example screenshots of the smartphone app are shown in FIG. 11 for the task of V_{T} estimation with calibration via IS.

FIG. 11A shows the main menu of the Android® app.

FIG. 11B shows the settings screen for the calibration maneuver with IS which allows the user to adjust the number of breaths and corresponding target volumes in IS.

FIG. 11C shows an example of the calibration model computed from the breathing data through an IS. Once the calibration model is computed, it is stored for further measurement Of V_{T}.

FIG. 11D shows an example of calibrated V_{T} estimates from the smartphone's chest movement signal, where the figure on top displays the processed waveform and detected breath phase onsets, and the figure at the bottom displays the corresponding V_{T} estimates during inspiratory phases. The average RR and average V_{T} are also displayed on this screen.

FIG. 11A-D show screenshots of the Android® smartphone application prototype for tidal volume estimation using the camera and calibration with an incentive spirometer (IS). FIG. 11A: Main menu of the Android® application. FIG. 11B: Calibration setup which allows adjustment of number of inspirations and target volumes. FIG. 11C: Example of calibration model computed while the subject breathed through the IS. Red dots indicate the first IS target (250 mL) and white dots the second target (500 mL). FIG. 11D: Example of tidal volume estimates after calibration. The top waveform indicates the chest movement signal from the smartphone camera. The bottom graph displays the estimated tidal volume of each inspiration. Average respiratory rate and tidal volume are also displayed.

TABLE 1
Tidal volume estimation results from smartphone-acquired
signals compared to the reference volume from the spirometer
(N = 12 subjects).

| | | Linear regression of | Calibration of smartphone |
|---|---|---|---|
| Parameter | | smartphone data | data using IS |
| RMSE | [L] | 0.147 ± 0.044 | 0.189 ± 0.074 |
| NRMSE | [%] | 14.499 ± 4.255 | 18.559 ± 6.579 |

Values presented as mean ± standard deviation

4. Discussion and Conclusions

Compared to a study (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press) that proposed the estimation of V_{T} directly on a smartphone by processing video recording information to obtain a chest movement signal correlated with reference V_{T} from a spirometer, the novel aspects according to embodiments disclosed herein include: 1) the introduction of an easy calibration procedure based just on the chest wall movement information recorded using the smartphone camera while breathing a few times through an inexpensive incentive spirometer device, 2) the full implementation of the signal processing methods on a smartphone app which makes it now possible for subjects to wirelessly control the calibration and measurement of tidal volume by themselves. Here, embodiments disclosed herein innovate a simple and attainable calibration procedure to easily allow V_{T} estimation on a daily basis without the use of specialized devices, e.g., spirometer. To calibrate the data from the smartphone-acquired signal, embodiments disclosed herein may use a widely-available volumetric incentive spirometer, of the sort that patients are often sent home with after hospitalization for a surgery. A smartphone application according to embodiments disclosed herein was implemented on an HTC One M8 Android® smartphone which allows recording of the chest movement signal, its calibration, and final V_{T} estimation. Performance of embodiments disclosed herein were tested by simultaneously recording a reference volume signal from a spirometer.

In another study (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press), it was found that the peak-to-peak amplitude of the smartphone-acquired chest movement signal is highly linearly correlated to tidal volume as measured by a spirometer system for twelve healthy volunteers. When the linear regression equation was used to normalize the smartphone data to tidal volume estimates, it was found an RMSE of 0.147±0.044 L which corresponded to a NRMSE of 14.499±4.255% when normalized to the mean value of the reference V_{T}. In turn, these errors were not statistically significantly different from those found in (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press) At this stage, the method for V_{T} estimation using a smartphone's camera has provided an average error of approximately 15% when calibrated using spirometry data as reference. However, it should be noted for normal ranges of tidal volume (−400-500 mL), the absolute error value is smaller than for the high tidal volume range (>1.5 L) as seen in FIGS. 8, 9 and 10. Note also that, as with other non-contact optical breathing monitoring methods, the calibration and tidal volume estimation results will be affected by, among other factors, the distance from and body angle of the subject with respect to the smartphone's camera.

Regarding wearing different types of clothes, the subjects were allowed to wear any pattern, e.g. plain, dotted, stripes, and colors of their clothing during the maneuvers. No significant difference in the results was noticed with different types of clothes. However, it was noticed that the quality of the signal decreased when clothes with smaller dots or prints were worn. Some recordings were also performed from subjects with bare skin and good data was still obtained.

A limitation of (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press) was noted in that the approach relied on being calibrated using a spirometer device; this specialized device is not commonly available outside research and clinical settings. In order to deal with this calibration restriction, embodiments disclosed herein may take advantage of the highly linear correlation between the chest movement signal and reference tidal volumes, to obtain a linear calibration model using only two sets of data points gathered while the volunteers breathed through an IS.

The IS device is a cheap device that is currently widely used in practice in many hospitals and nursing homes for the purpose of rebuilding diaphragm muscles for those subjects who have been on a respirator or immobilized for several days due to surgery. Each data set consisted of the peak-to-peak amplitudes of the chest movement signal during inspirations at 250 mL and 500 mL targets marked on the IS. To minimize the effect of a possible outlier when breathing at IS targets, the median value of each data set was taken as representative to compute the calibration model. It was found that when calibrated using the linear model from the first IS maneuver, the smartphone-based V_{T} estimation provided a RMSE of 0.189±0.074 L equivalent to 18.559±6.579% when normalized. This error represents a statistically-significant increment of around 4% compared to the NRMSE error obtained from calibration using a spirometer. Also, in contrast to the V_{T} estimation obtained from calibration via spirometer, it was found that a statistically-significant fixed bias of −51 mL when the calibration was performed using data from the IS maneuver. This higher estimation error and systematic V_{T} underestimation could be attributable to limitations of the IS which does not offer a more precise estimation of the inspired volume due to its coarse volume scale as well as to the increase in airway resistance when using it, which in turn increases the chest movements due to a higher breathing effort and hence shifting of the peak-to-peak of chest movement signal to higher values from which the calibration model is constructed. Some of the estimation error can be attributed to the fact that when the IS was calibrated using a calibration syringe and it was found that the former is off by ˜2-3% when compared to the latter. Moreover, it should be noted that despite the best attempts by the subjects to hit the predefined target volumes, they often either under or over-achieved the target volume. Besides these performance degradations, and even when calibration should be performed on-site prior to estimating V_{T} for a given breathing maneuver, the calibration method is easy-to-perform and does not employ a specialized nor expensive device. The calibration procedure itself, both maneuver and calculation takes less than 30 seconds and is automatically performed by the smartphone app, with the option to be remotely started and completed via a wireless controller. Note, however, that the method may use individualized calibration prior to tidal volume measurement using a smartphone camera. Hence, it is necessary for subjects to be familiar with the calibration procedure and the correct use of the IS in order to minimize estimation errors.

According to embodiments disclosed herein, deployment of an incentive spirometer enables an individualized calibration procedure to be performed, and, hence, enables the V_{T} estimation in everyday settings. Taking into account the behavior of the estimation error at the different volume levels, as shown in FIG. 10, where the dispersion of the smartphone-based estimates increases at high volume levels, as well as by considering the use of the IS device to improve patients' breathing after surgery, it may be envisioned that subjects may use methods and apparatus according to embodiments disclosed herein at their homes to estimate the progress in their V_{T} recovery. The person would place their smartphone at a fixed location, stand still in front of it and conduct a series of breathing routines to obtain some volume estimates. By doing so, this would also minimize the motion artifacts.

Some observations may be made. First, breathing data was collected while the healthy subjects were standing still, i.e., performance of the V_{T} estimation during motion, postural changes, and airway obstruction was not explored. Second, a low number of subjects were tested. A future study involving a larger sample size with different age categories as well as balanced gender groups may be useful. Third, only a limited area of the anterior chest wall was monitored, i.e., the rib cage area using a rectangular ROI, and this could ignore small contributions of other compartments and anatomical distortions in other areas. As such, exploration of these topics may be useful as well as other topics.

In particular, there is interest in the implementation of methods to deal with body motion artifacts, as proposed in the literature for RR monitoring (Shao, D.; Yang, Y.; Liu, C.; Tsow, F.; Yu, H.; Tao, N. Noncontact Monitoring Breathing Pattern, Exhalation Flow Rate and Pulse Transit Time. IEEE Trans. Biomed. Eng. 2014, 61, 2760-2767; Sun, Y.; Hu, S.; Azorin-Peris, V.; Greenwald, S.; Chambers, J.; Zhu, Y. Motion-compensated noncontact imaging photoplethysmography to monitor cardiorespiratory status during exercise. J. Biomed. Opt. 2011, 16, 077010-077010). The implementation of image processing techniques to monitor a ROI beyond a simple rectangular area is also pending work. Note however that scenarios including motion artifacts are less likely to occur when measuring V_{T} during a short maneuver but it would become an important issue if continuous monitoring is intended. In addition, analyzing the performance of the smartphone-based estimator in different postures including supine, when the abdominal mechanical degree of freedom is expected to dominate the contribution to V_{T}, is pending. The analysis of the performance of the tidal volume estimation method at different levels of illumination, distance from and angle of the subject's thoracic area with respect to the smartphone's camera are other pending topics to be explored in the future. Other applications for the developed smartphone-based monitor in the area of respiratory sound analysis may be explored where a temporal reference would be helpful to classify and characterize the recorded sounds, particularly in patients presenting adventitious respiratory sounds.

The development of an inexpensive and portable breathing monitoring system for on-demand V_{T} and RR estimation capabilities is still pending for the general population. The near-ubiquity of smartphones and their owners' high reliance on them makes them an attractive alternative to develop a system with those characteristics. Although several advances have been made regarding cardiac monitoring using smartphones, a limited number of studies have addressed their applications to respiratory monitoring, and most of them have focused on respiratory rate estimation despite the importance of monitoring respiratory depth. The results stemming from embodiments disclosed herein support the feasibility of developing a smartphone-based breathing monitor that provides V_{T} estimates when calibrated using a simple, affordable, and widely-accessible external device. Development of such a system according to embodiments disclosed herein would advance on-demand monitoring by providing another breathing parameter in addition to the number of breaths-per-minute.

Tidal Volume and Instantaneous Respiration Rate Estimation Using a Volumetric Surrogate Signal Acquired via a Smartphone Camera

As disclosed above, two parameters that a breathing status monitor may provide include tidal volume (V_{T}) and respiration rate (RR). An optical monitoring method according to embodiments disclosed herein that tracks chest wall movements was implemented directly on a smartphone. Embodiments disclosed herein may make use of such noncontact optical monitoring to obtain a volumetric surrogate signal, via analysis of intensity changes in the video channels caused by the chest wall movements during breathing, in order to provide not just average RR, but also information about V_{T} and to track RR at each time-instant (IRR).

The method, implemented on an Android® smartphone, was used to analyze the video information from the smartphone's camera and provide in real time the chest movement signal from N=15 healthy volunteers each breathing at V_{T} ranging from 300 mL to 3 L. These measurements were performed separately for each volunteer. Simultaneous recording of volume signals from a spirometer was regarded as reference. A highly linear relationship between peak-to-peak amplitude of the smartphone-acquired chest movement signal and spirometer V_{T} was found (r²=0.951±0.042, mean±SD). After calibration on a subject-by-subject basis, no statistically-significant bias was found in terms of V_{T} estimation; the 95% limits of agreement were −0.348 to 0.376 L, and the RMSE was 0.182±0.107 L. In terms of IRR estimation, a highly linear relation between smartphone estimates and the spirometer reference was found (r²=0.999±0.002). The bias, 95% limits of agreement, and RAISE were −0.024 bpm, −0.850 to 0.802 bpm, and 0.414±0.178 bpm, respectively. These promising results show the feasibility of developing an inexpensive and portable breathing monitor which could provide information about IRR as well as V_{T}, when calibrated on an individual basis according to embodiments disclosed herein using, for example, smartphones.

I. Introduction

Monitoring of respiration status has been recognized as critical to identifying and predicting serious adverse events (F. Q. Al-Khalidi, R. Saatchi, D. Burke, H. Elphick, and S. Tan, “Respiration rate monitoring methods: A review,” Pediatr. Pulmonol., vol. 46, no. 6, pp. 523-529, Jun. 2011; M. A. Cretikos, R. Bellomo, K. Hillman, J. Chen, S. Finfer, and A. Flabouris, “Respiratory rate: the neglected vital sign,” Med. J. Aust., vol. 188, no. 11, p. 657, 2008). Two basic parameters that a breathing monitor should be able to provide are tidal volume (VT) and respiration rate (RR) (M. Folke, L. Cernerud, M. Ekström, and B. Hök, “Critical review of non-invasive respiratory monitoring in medical care,” Med. Biol. Eng. Comput., vol. 41, no. 4, pp. 377-383, July 2003). V_{T} provides information about the respiration depth and is defined as the volume of air moved with each breath; on the other hand, RR corresponds to the number of breaths per unit of time and is commonly expressed in breaths-per-minute. In turn, the product of these two quantities defines the volume of gas moved by the respiratory system per minute, called minute ventilation (\dot{V}_{E}). Normal average values for a human are around 0.5 L and 12 breaths-per-minute (bpm) for V_{T} and RR, respectively. These values are not fixed and the mechanism of respiratory control is crucial in determining \dot{V}_{E} by adjusting the combination of V_{T} and RR according to a body's requirements in response to different scenarios (B. M. Koeppen and B. A. Stanton, Berne & Levy Physiology, Updated Edition. Elsevier Health Sciences, 2009).

Current clinical continuous RR monitoring methods include qualified human observation, transthoracic impedance, inductance plethysmography, capnography monitoring, and tracheal sound monitoring (K. P. Cohen, W. M. Ladd, D. M. Beams, W. S. Sheers, R. G. Radwin, W. J. Tompkins, and J. G. Webster, “Comparison of impedance and inductance ventilation sensors on adults during breathing, motion, and simulated airway obstruction,” IEEE Trans. Biomed. Eng., vol. 44, no. 7, pp. 555-566, July 1997; G. B. Drummond, A. F. Nimmo, and R. A. Elton, “Thoracic impedance used for measuring chest wall movement in postoperative patients.,” Br. J. Anaesth., vol. 77, no. 3, pp. 327-332, September 1996; M. A. E. Ramsay, M. Usman, E. Lagow, M. Mendoza, E. Untalan, and E. De Vol, “The Accuracy, Precision and Reliability of Measuring Ventilatory Rate and Detecting Ventilatory Pause by Rainbow Acoustic Monitoring and Capnometry:,” Anesth. Analg., vol. 117, no. 1, pp. 69-75, July 2013; J. J. Vargo, G. Zuccaro Jr., J. A. Dumot, D. L. Conwell, J. B. Morrow, and S. S. Shay, “Automated graphic assessment of respiratory activity is superior to pulse oximetry and visual assessment for the detection of early respiratory depression during therapeutic upper endoscopy,” Gastrointest. Endosc., vol. 55, no. 7, pp. 826-831, June 2002). Each method has its own disadvantages, e.g. it is time consuming and subjective to do human observation, patients have a low tolerance for using the nasal cannula in capnography (M. Folke, L. Cernerud, M. Ekström, and B. Hök, “Critical review of non-invasive respiratory monitoring in medical care,” Med. Biol. Eng. Comput., vol. 41, no. 4, pp. 377-383, July 2003). However flawed, at least clinical devices exist for monitoring. Outside clinical or research settings, there is still a lack of monitoring devices that can very accurately determine RR in a non-invasive way, to be used on a daily basis.

Regarding V_{T} measurement, current clinical methods include spirometry, impedance pneumography, inductance plethysmography, photoplethysmography, computed tomography, phonospirometry, Doppler radar, and more recently electrocardiography (K. Ashutosh, R. Gilbert, J. H. Auchincloss, J. Erlebacher, and D. Peppi, “Impedance pneumograph and magnetometer methods for monitoring tidal volume,” J Appl Physiol, vol. 37, no. 6, pp. 964-966, 1974; P. Grossman, M. Spoerle, and F. H. Wilhelm, “Reliability of respiratory tidal volume estimation by means of ambulatory inductive plethysmography,” Biomed. Sci. Instrum., vol. 42, pp. 193-198, 2006; A. Johansson and P. P. Å. Öberg, “Estimation of respiratory volumes from the photoplethysmographic signal. Part I: experimental results,” Med. Biol. Eng. Comput., vol. 37, no. 1, pp. 42-47, January 1999; Y. S. Lee, P. N. Pathirana, C. L. Steinfort, and T. Caelli, “Monitoring and Analysis of Respiratory Patterns Using Microwave Doppler Radar,” IEEE J. Transl. Eng. Health Med., vol. 2, pp. 1-12, 2014; G. Li, N. C. Arora, H. Xie, H. Ning, W. Lu, D. Low, D. Citrin, A. Kaushal, L. Zach, K. Camphausen, and R. W. Miller, “Quantitative prediction of respiratory tidal volume based on the external torso volume change: a potential volumetric surrogate,” Phys. Med. Biol., vol. 54, no. 7, pp. 1963-1978, April 2009; M. R. Miller, J. Hankinson, V. Brusasco, F. Burgos, R. Casaburi, A. Coates, R. Crapo, P. Enright, C. P. M. van der Grinten, P. Gustafsson, and others, “Standardisation of spirometry,” Eur. Respir. J., vol. 26, no. 2, pp. 319-338, 2005; C.-L. Que, C. Kolmaga, L.-G. Durand, S. M. Kelly, and P. T. Macklem, “Phonospirometry for noninvasive measurement of ventilation: methodology and preliminary results,” J. Appl. Physiol. Bethesda Md 1985, vol. 93, no. 4, pp. 1515-1526, October 2002; O. Sayadi, E. H. Weiss, F. M. Merchant, D. Puppala, and A. A. Armoundas, “An Optimized Method for Estimating the Tidal Volume from Electrocardiographic Signals: Implications for Estimating Minute Ventilation,” Am. J. Physiol.—Heart Circ. Physiol., vol. 307, pp. H426-H436, 2014; B. J. Semmes, M. J. Tobin, J. V. Snyder, and A. Grenvik, “Subjective and objective measurement of tidal volume in critically ill patients.,” Chest, vol. 87, no. 5, pp. 577-579, 1985). Similar to RR estimation, limitations arise when estimating V_{T}, e.g. high doses of ionizing radiation in computed tomography, or alteration in both natural RR and V_{T} due to spirometer use (R. Gilbert, J. H. Auchincloss, J. Brodsky, and W. Boden, “Changes in tidal volume, frequency, and ventilation induced by their measurement.,” J. Appl. Physiol., vol. 33, no. 2, pp. 252-254, Aug. 1972). Moreover, having been designed for clinical settings or research centers, these methods employ specialized devices that are not translated easily to everyday use due to their high costs, need for skilled operators, or limited mobility.

Smartphones have become widely available and vital sign applications have been found to be accurate and robust. In addition, smartphones have fast microprocessors, large data storage and media capabilities which make them an enticing option for developing a ubiquitous mobile respiration monitoring system. In an attempt to develop such a mobile system, an acoustical approach was analyzed and good correlation was found between the smartphone-based respiration rate estimates and the spirometer-based ones (r²≈0.97), as well as 95% limits of agreement ranging approximately from −1.4 to 1.6 bpm for a breathing range from 15 to 35 bpm (B. A. Reyes, N. Reljin, and K. H. Chon, “Tracheal Sounds Acquisition Using Smartphones,” Sensors, vol. 14, no. 8, pp. 13830-13850, July 2014). However, the last approach requires plugging an additional acoustical sensor into the smartphone in order to extract information from tracheal sounds and just provides estimates of RR and breath-phase onset.

In order to overcome the need for an external sensor for the task of RR estimation, i.e., the acoustical sensor, embodiments disclosed herein may take advantage of a smartphone's cameras. In particular, a method according to embodiments disclosed herein allows the real-time acquisition of a surrogate volumetric signal from breathing-related light intensity changes due to chest wall movements was implemented on a smartphone and its performance and was tested in healthy volunteers breathing at a metered pace and spontaneously, while seated. Under the paced breathing, it was found that the smartphone-based estimates of average RR were accurate when compared to those obtained from inductance plethysmography.

In general, a noncontact optical breathing monitor employs a video camera placed at distance from the subject's body to capture the intensity changes of the reflected light caused by his/her chest wall movements as they modify the path length of the illumination light (F. Zhao, M. Li, Y. Qian, and J. Z. Tsien, “Remote Measurements of Heart and Respiration Rates for Telemedicine,” PLoS ONE, vol. 8, no. 10, p. e71384, October 2013). These chest wall movements also change the amount of light reflected back to the video camera. During inspiration, the inspiratory muscles contract, resulting in an enlarged thoracic cavity; the diaphragm descends downward increasing the vertical dimension while the external intercostal muscles elevate the ribs and move the sternum upward and outward increasing the thoracic cavity in the horizontal axis. Due to this contraction the lungs expand to fill the larger thoracic cavity, resulting in a drop of the intra-alveolar pressure that causes a flow of air into the lungs until the intra-alveolar pressure equals the atmospheric pressure (L. Sherwood, Fundamentals of Human Physiology, 4th ed. Boston, Mass., USA: Cengage Learning, 2011). The inspiratory muscles relax during the expiration, restoring the chest wall and stretched lungs to their preinspiratory sizes, due to their elastic properties, and causing a rise in the intra-alveolar pressure above atmospheric level forcing the air to leave the lungs (L. Sherwood, Fundamentals of Human Physiology, 4th ed. Boston, Mass., USA: Cengage Learning, 2011). Note that in the noncontact optical respiratory monitoring approach, volume changes are not directly measured but a surrogate signal is obtained from the analysis of the variations in the reflected light due to chest wall movements captured by the system's camera while breathing.

There have been efforts to perform respiratory monitoring via the noncontact optical approach described above, but most of them have solely focused on average RR estimation F. Zhao, M. Li, Y. Qian, and J. Z. Tsien, “Remote Measurements of Heart and Respiration Rates for Telemedicine,” PLoS ONE, vol. 8, no. 10, p. e71384, October 2013; M. Bartula, T. Tigges, and J. Muehlsteff, “Camera-based system for contactless monitoring of respiration,” in 2013 35th Annual International Conference of the IEEE Engineering in Medicine and Biology Society (EMBC), 2013, pp. 2672-2675; S. J. Cala, C. M. Kenyon, G. Ferrigno, P. Carnevali, A. Aliverti, A. Pedotti, P. T. Macklem, and D. F. Rochester, “Chest wall and lung volume estimation by optical reflectance motion analysis,” J. Appl. Physiol., vol. 81, no. 6, pp. 2680-2689, December 1996; M.-Z. Poh, D. J. McDuff, and R. W. Picard, “Advancements in Noncontact, Multiparameter Physiological Measurements Using a Webcam,” IEEE Trans. Biomed. Eng., vol. 58, no. 1, pp. 7-11, January 2011; D. Shao, Y. Yang, C. Liu, F. Tsow, H. Yu, and N. Tao, “Noncontact Monitoring Breathing Pattern, Exhalation Flow Rate and Pulse Transit Time,” IEEE Trans. Biomed. Eng., vol. 61, no. 11, pp. 2760-2767, November 2014; L. Tarassenko, M. Villarroel, A. Guazzi, J. Jorge, D. A. Clifton, and C. Pugh, “Non-contact video-based vital sign monitoring using ambient light and auto-regressive models,” Physiol. Meas., vol. 35, no. 5, p. 807, 2014; H.-Y. Wu, M. Rubinstein, E. Shih, J. Guttag, F. Durand, and W. Freeman, “Eulerian Video Magnification for Revealing Subtle Changes in the World,” ACM Trans Graph, vol. 31, no. 4, pp. 65:1-65:8, July 2012). Still, noncontact optical methods have been proposed for VT estimation, which is more challenging than average RR estimation. In particular, chest wall surface markers tracked by an optical reflectance system have shown promising results (S. J. Cala, C. M. Kenyon, G. Ferrigno, P. Carnevali, A. Aliverti, A. Pedotti, P. T. Macklem, and D. F. Rochester, “Chest wall and lung volume estimation by optical reflectance motion analysis,” J. Appl. Physiol., vol. 81, no. 6, pp. 2680-2689, December 1996). Those findings have been supported by studies that showed a one-to-one relationship between changes of the external torso and V_{T} corresponding to internal lung air content (G. Li, N. C. Arora, H. Xie, H. Ning, W. Lu, D. Low, D. Citrin, A. Kaushal, L. Zach, K. Camphausen, and R. W. Miller, “Quantitative prediction of respiratory tidal volume based on the external torso volume change: a potential volumetric surrogate,” Phys. Med. Biol., vol. 54, no. 7, pp. 1963-1978, April 2009). More recently, a webcam and image processing technique based on the detection of shoulder displacements were implemented for breathing pattern tracking (D. Shao, Y. Yang, C. Liu, F. Tsow, H. Yu, and N. Tao, “Noncontact Monitoring Breathing Pattern, Exhalation Flow Rate and Pulse Transit Time,” IEEE Trans. Biomed. Eng., vol. 61, no. 11, pp. 2760-2767, November 2014).

Observation of smartphone-acquired signals pointed to the possibility of obtaining more valuable information than the average RR. Namely, a development of method according to embodiments disclosed herein capable of monitoring the increased amplitude of the chest movements when volunteers took deeper breaths.

According to embodiments disclosed herein, a mobile system based on a noncontact optical approach implemented in a smartphone that provides information, from a volume surrogate, about both RR at each time instant (IRR) as well as V_{T} (when calibrated), in contrast to just average RR. According to embodiments disclosed herein, a respiratory monitoring system may be implemented on a commercially-available Android® smartphone, but could of course be implemented in smartphones using other operating systems. Signals were collected from healthy volunteers and the performance of the smartphone system for the tasks of IRR and V_{T} estimation was tested, using the spirometer-acquired volume signal as reference.

II. Materials And Methods

A. Subjects

For this study, fifteen (N=15) healthy and non-smoker volunteers (fourteen males and one female) aged 19 to 52 years (mean±standard deviation: 28.73±9.27), weight 70.14±19.83 kg and height 175.67±5.94 cm, were recruited. Exclusion criteria included individuals with previous pneumothorax, those with chronic respiratory illnesses such as asthma, and anyone who was currently ill with the common cold or an upper respiratory infection. The group of volunteers consisted of students and staff members from the University of Connecticut (UConn), USA. Each volunteer consented to be a subject and signed the study protocol approved by the Institutional Review Board of UConn.

B. Respiration Signals Acquisition

Equipment

The HTC One M8 smartphone (HTC Corporation, New Taipei City, Taiwan) running the Android® v4.4.2 (KitKat) operating system was selected for this research as it is one of the state-of-the-art Android® smartphones which is nowadays the dominant operating system worldwide in mobile devices. The HTC One M8 allows simultaneous dual camera recording supported by its processor running a 2.3 GHz quad-core CPU (Snapdragon 801, Qualcomm Technologies Inc., San Diego, Calif., USA). For this study, the chest movement signal of interest was collected via the frontal camera consisting of a 5 MP, backside-illumination sensor with wide angle lens and 1080p full HD video recording capabilities at 30 frames-per-second. The video recording was processed in real time using an application specifically designed for and implemented in the smartphone to obtain a volumetric surrogate signal, referred to in this paper as the chest movement signal, of the subject as discussed in the next section. After finishing the maneuver, the chest movement signal and corresponding time vector were saved into a text file in the smartphone and transferred to a personal computer for offline analysis of results using Matlab® (R2012a, The Mathworks, Inc., Natick, Mass., USA).

Together with the smartphone-recorded volumetric surrogate signal, a spirometer system consisting of a respiration flow head connected to a differential pressure transducer to measure airflow was used to record the airflow signal (MLT1000L, FE141 Spirometer, ADlnstruments, Inc., Dunedin, New Zealand). The volume signal, regarded as reference for V_{T} and IRR estimation, was computed in the phone as the integral of the airflow over time. Both the airflow and volume signals were sampled at 1 kHz using a 16-bit A/D converter (PowerLab/4SP, ADlnstruments, Inc., Dunedin, New Zealand). A 3.0 L calibration syringe (Hans Rudolph, Inc., Shawnee, Kans., USA) was used to calibrate the spirometer system prior to recording of each volunteer. A new set consisting of disposable filter, reusable mouthpiece, and disposable nose clip was given to each volunteer (MLA304, MLA1026, MLA1008, ADlnstruments, Inc., Dunedin, New Zealand).

Acquisition Protocol

Each maneuver lasted approximately 2 minutes during which the volunteers were asked to breathe through the spirometer system at different volume levels ranging from around 300 mL to 3 L depending on what was manageable for that individual. Each subject was instructed to breathe while first increasing their V_{T} with each breath for around 1 minute, and then decreasing their V_{T} with each breath for the remaining time. To provide visual feedback of the maneuver to the volunteers, their volume signal was displayed on a 40″ monitor placed in front of them. Nose clips were used to clamp the nostrils during the respiration maneuver. Subjects were standing still during signal collection. The smartphone was positioned in front of the subject at approximately 60 cm in a 3-pronged clamp placed at thorax level so that the frontal camera recorded chest wall movements associated with breathing during the maneuver. All signals were recorded in a regular dry lab with the ambient light which predominantly consisted of ordinary fluorescent lamps located in the ceiling approximately 2.5 m above floor level and to a lesser extent, sunlight entering through the lab's windows. Although the smartphone and spirometer recordings were simultaneously started, 5 seconds of initial and final apnea segments were acquired for automatic alignment purposes between both recordings. After initial apnea, subjects took a forced respiration cycle before performing the described respiration maneuver.

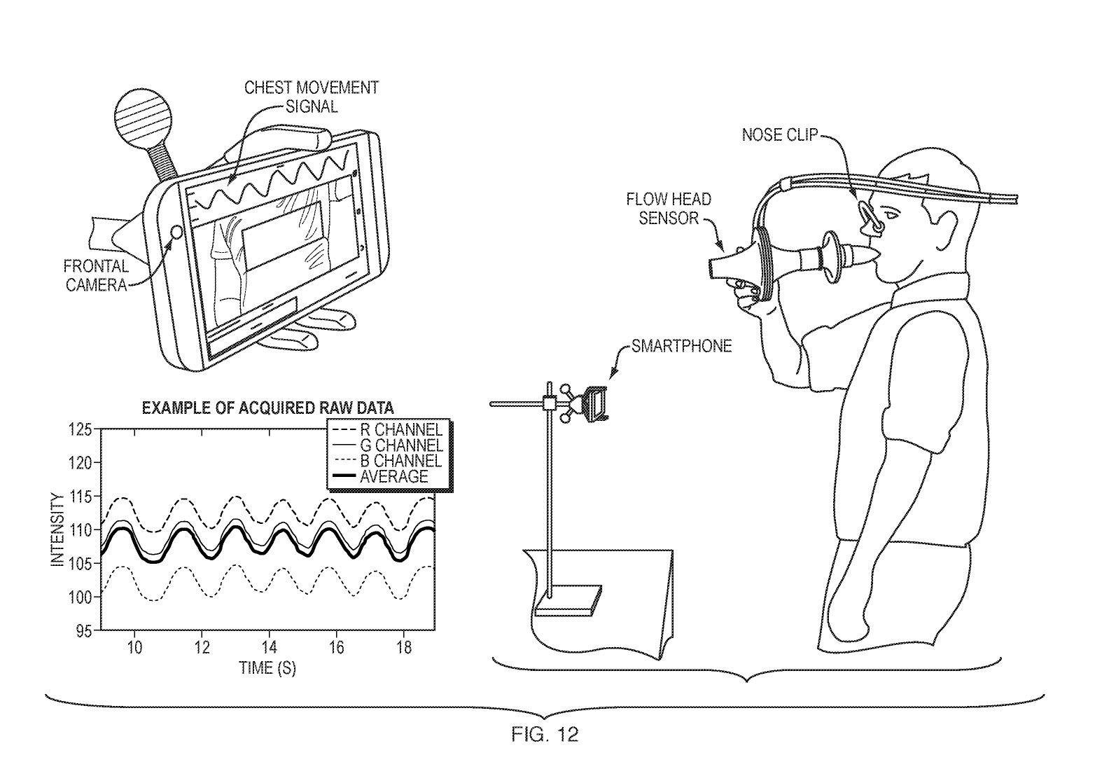

FIG. 12 is an example of the experimental setup. It is worth mentioning that volunteers were not restricted in wearing any color/pattern of their clothes during the maneuvers but instructed not to wear loose clothes.

C. Chest Movement Recording Method

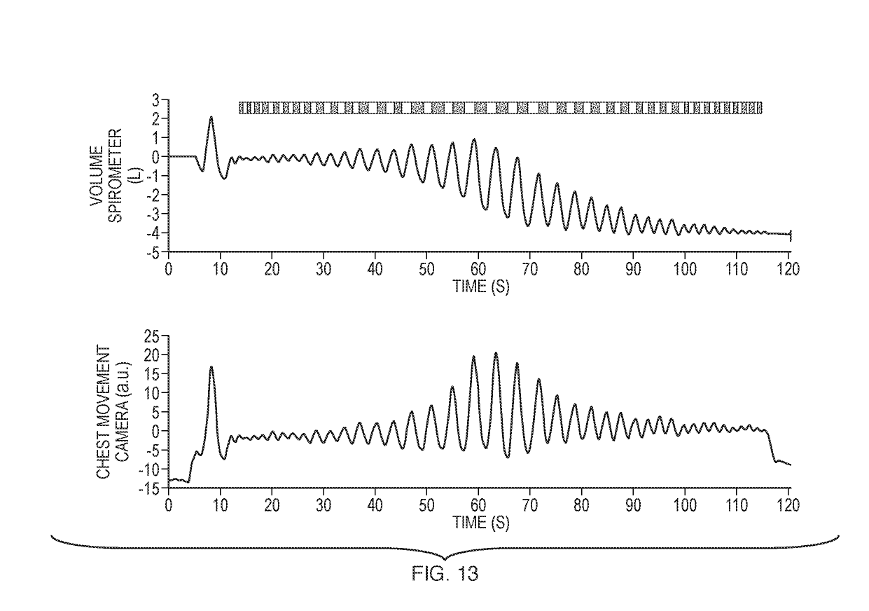

The two major anatomical contributors to the visibility of breathing are the rib cage and abdomen compartments of the chest wall, whose movements in the anteroposterior direction are greater than those in the vertical or transverse directions, with an increase of around 3 cm in the anteroposterior diameter over the vital capacity range (K. Konno and J. Mead, “Measurement of the separate volume changes of rib cage and abdomen during breathing,” J. Appl. Physiol., vol. 22, no. 3, pp. 407-423, March 1967). There is a relationship between volume displacement and linear motion during breathing (K. Konno and J. Mead, “Measurement of the separate volume changes of rib cage and abdomen during breathing,” J. Appl. Physiol., vol. 22, no. 3, pp. 407-423, March 1967), and a one-to-one relationship between changes of the external torso and tidal volume corresponding to internal lung air content has been found (G. Li, N. C. Arora, H. Xie, H. Ning, W. Lu, D. Low, D. Citrin, A. Kaushal, L. Zach, K. Camphausen, and R. W. Miller, “Quantitative prediction of respiratory tidal volume based on the external torso volume change: a potential volumetric surrogate,” Phys. Med. Biol., vol. 54, no. 7, pp. 1963-1978, April 2009). Embodiments of the smartphone method may be intended to take advantage of this relationship to obtain a volumetric surrogate by analyzing the changes in the intensity of the reflected light caused by the breathing-related chest wall movements captured at a distance with a smartphone's camera. In particular, the method processes video recordings in real time, where at each time instant t, the intensities of the red, green and blue (RGB) channels are averaged within a rectangular region of interest (ROI) according to

$$I ( t ) = ( \frac{1}{3 D} ) ( {\sum }_{\{ m , n \} ∈ ROI} {i}_{R} ( m , n , t ) + {\sum }_{\{ m , n \} ∈ ROI} {i}_{G} ( m , n , t ) + {\sum }_{\{ m , n \} ∈ ROI} {i}_{B} ( m , n , t ) ) \qquad ( 1 )$$

where iₓ(m,n,t) is the intensity value of the pixel at the m-th row and n-th column of the red, green or blue channel within the ROI containing a total of D pixels. For this study, a region of 49×90 pixels were selected in a resolution of 320×240 pixels and focused on the thoracic area of the subject. This reduced resolution and ROI size were selected so that they do not compromise the sampling rate during the real time monitoring in the smartphone app. With these settings, the frame rate dropped to around 25 frames-per-second. The average intensity waveform I (t) was regarded as the chest movement signal, i.e., the volume surrogate, from which the tidal volume and respiratory rates were estimated. As shown in FIG. 12, despite the DC values, all channels carry similar information, and hence their average was taken to avoid channel selection. An example of the raw volume acquired with a spirometer and the corresponding chest movement signal acquired online with the smartphone's camera and chest movement app is shown in FIG. 13 for the respiration maneuver performed by one subject. It should be noted that similar to other monitoring methods, e.g. inductance plethysmography, the proposed noncontact optical approach via the smartphone-acquired volumetric surrogate signal might be very weak if the clothes worn by the subject are not tight to his/her thorax, which can result in increased estimation errors of breathing parameters.

D. Data Preprocessing

The acquired chest movement signal was interpolated at 25 Hz via a cubic spline method to achieve a uniform sampling rate that corrects fluctuations around this value during the online acquisition in the smartphone. The reference volume signal was down-sampled to 25 Hz to achieve the same sampling frequency as the chest movement signal. In order to minimize high frequency components not related to the respiration maneuver, the chest movement and reference volume signals were filtered with a 4th-order Butterworth lowpass filter at 2 Hz that was applied in a forward and backward scheme to produce zero-phase distortion and minimize the start and end transients.

After filtering, the chest movement and reference volume signals were automatically aligned using the cross-correlation function, where 20 seconds in the central portion of the maneuver were extracted from each recording to compute the cross-correlation sequence in order to obtain the sample lag providing the maximum cross-correlation value that indicates the required samples to be shifted. This alignment was required because of different starting times and delays of the smartphone and AD converter acquisition systems during the simultaneous recording of the maneuver. The duration of the signals was set accordingly, to the minimum duration of both types of recordings.

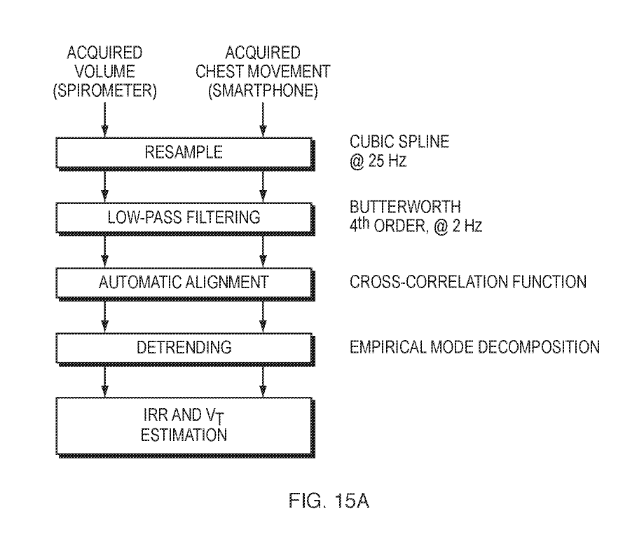

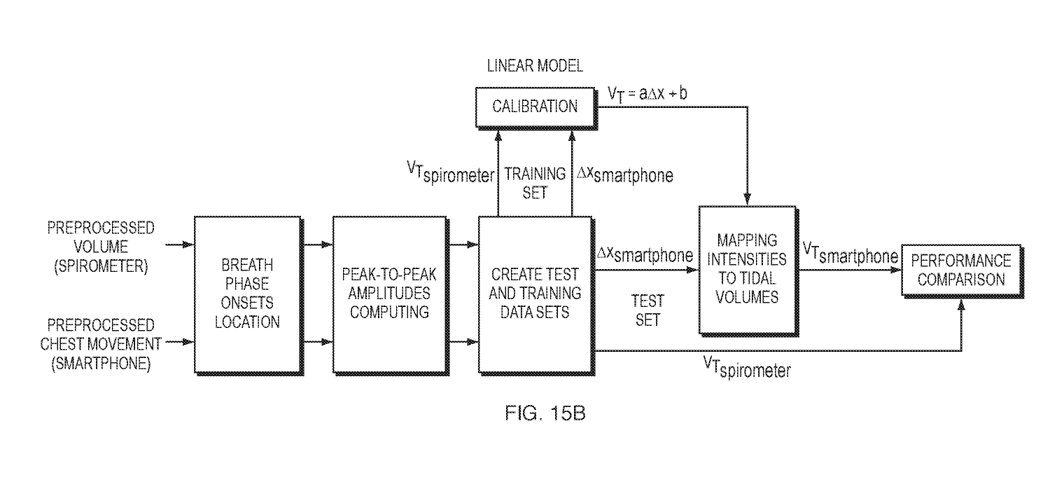

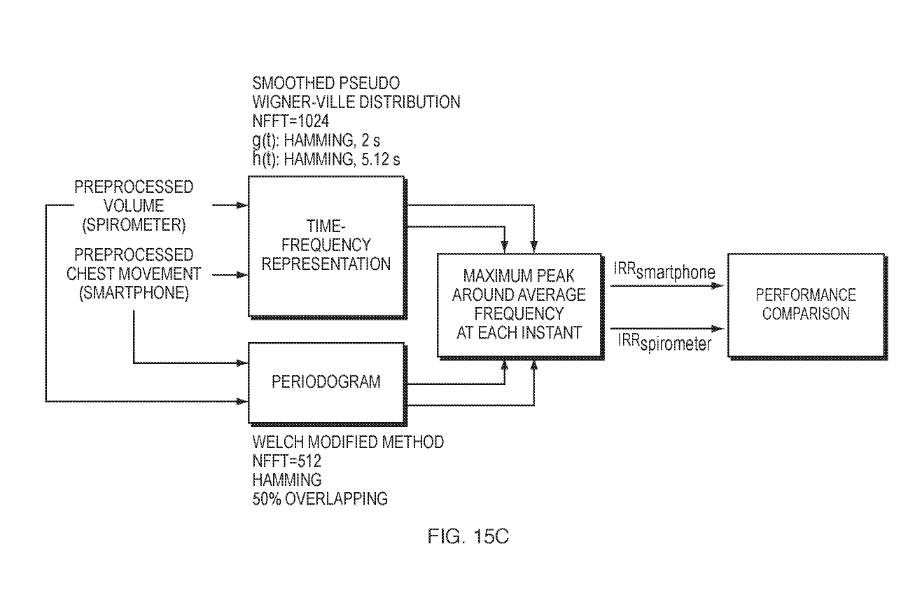

Finally, both signals, the surrogate and actual volume, were detrended via the Empirical Mode Decomposition (EMD) method (N. E. Huang, Z. Shen, S. R. Long, M. C. Wu, H. H. Shih, Q. Zheng, N.-C. Yen, C. C. Tung, and H. H. Liu, “The empirical mode decomposition and the Hilbert spectrum for nonlinear and non-stationary time series analysis,” Proc. R. Soc. Lond. Ser. Math. Phys. Eng. Sci., vol. 454, no. 1971, pp. 903-995, 1998). The essence of this decomposition is to identify the intrinsic oscillatory modes, called IMFs, of a signal through the time scales present in it. Its principal attractiveness resides in obtaining the IMFs directly from the signal without the use of any kernel, i.e., EMD depends only on the data. All the IMFs of the signal s(t) under analysis are extracted automatically by a shifting process intended to eliminate riding waveforms and to produce close to zero mean value as defined by upper and lower envelope signals. The EMD sifting process allows representation of the original signal in term of its extracted components as

$$s ( t ) = {\sum }_{k = 1}^{K} {IMF}_{k} ( t ) + {r}_{K} ( t ) \qquad ( 8 )$$

where K is the total number of IMFs, and r_{K}(t) is the residual signal. EMD has the characteristic of being a complete decomposition (N. E. Huang, Z. Shen, S. R. Long, M. C. Wu, H. H. Shih, Q. Zheng, N.-C. Yen, C. C. Tung, and H. H. Liu, “The empirical mode decomposition and the Hilbert spectrum for nonlinear and non-stationary time series analysis,” Proc. R. Soc. Lond. Ser. Math. Phys. Eng. Sci., vol. 454, no. 1971, pp. 903-995, 1998). As shown in FIG. 13, the acquired reference volume from the spirometer, and the chest wall movement signal from the smartphone camera, consist of a slowly varying trend superimposed on the fluctuating breathing signal of interest. As a result of the sifting process, the first IMFs contain the lower scales (higher frequency components), while the trend is contained in the last IMFs. Hence, the selection of the appropriate IMFs was based on the mean of the fine-to-coarse EMD reconstruction by observing the evolution of the empirical mean of the reconstructions as a function of the test order (K), and identifying the order at which it departs significantly from zero (P. Flandrin, P. Goncalves, and G. Rilling, “Detrending and denoising with empirical mode decomposition,” in Proceedings of the European signal processing conference (EUSIPCO′04), 2004, vol. 2, pp. 1581-1584). A flowchart of the signal preprocessing stage is shown in FIG. 15A.

E. Tidal Volume Estimation Using Smartphone Camera Signal

The volume signal from the spirometer was used to automatically determine the breath-phase onsets during the maneuver by finding their local maxima and minima. Inspiratory and expiratory phases corresponded to positive and negative traces of the volume signal, respectively. The V_{T} of each phase was computed as the absolute volume difference between two consecutive breath-phase onsets. The time location of the onsets was used to determine the corresponding maxima or minima in the aligned chest movement signal around a time window of 500 ms centered at each breath-phase onset. The amplitude difference between two consecutive breath-phase onsets in the chest movement signal was used for V_{T} estimation via the smartphone.

For calibration, a least-squares linear regression between the reference V_{T} and the absolute peak-to-peak amplitude of chest movement was performed for each subject; half of the data points of the maneuver were randomly selected for calibration purposes and regarded as a training data set, while the remaining half were used as a test data set to which the computed linear model was applied in order to map the smartphone-based measurements to volume estimates in liters.

The performance of the V_{T} estimation was measured on the test data using the regression parameter r², the root-mean-squared error RMSE, and the normalized root-mean-squared error NRMSE, defined as follows

$$\begin{matrix}RMSE = \sqrt{\frac{{\sum }_{i = 1}^{M} {( {V}_{{T}_{spirometer}} ( i ) - {V}_{{T}_{smartphone}} ( i ) )}^{2}}{M}} & ( 9 ) \\ NRMSE = \frac{RMSE}{mean ( {V}_{{T}_{spirometer}} )} \times 100 \% & ( 10 )\end{matrix}$$

where V_{T}_{ₛₚᵢᵣₒₘₑₜₑᵣ} indicates the tidal volume obtained from the spirometer-acquired volume signal, V_{T}_{ₛₘₐᵣₜₚₕₒₙₑ} the tidal volume estimated from smartphone-acquired chest movements after calibration, and M is the number of breath-phases of the analyzed maneuver used for testing. A flow chart of the tidal volume estimation stage is shown in FIG. 15B.

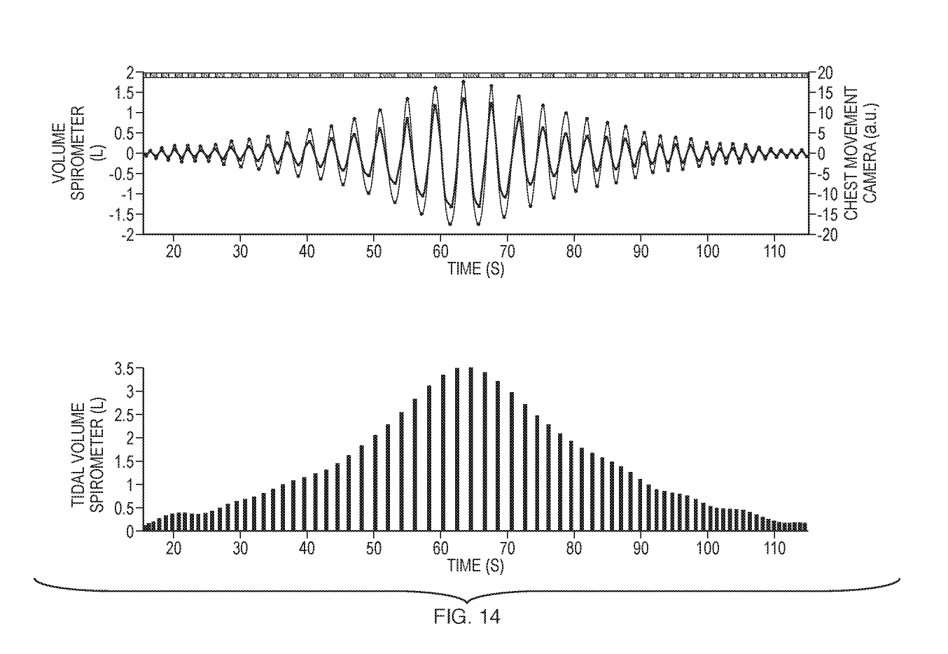

FIG. 14 shows an example of the preprocessed reference volume and chest movement signals. The breath-phase onsets and respiration phases as computed from the volume signal are indicated on top. The corresponding maxima and minima are superimposed on each signal. The detrended versions of the spirometer and smartphone signals shown in FIG. 13 are shown in FIG. 14, after applying the EMD approach. Note that although the inspiratory and expiratory phases of the maneuver can be noticed in both types of acquired signals as positive and negative segments in FIG. 13, the signal detrending stage simplifies their further processing. The corresponding V_{T} of each respiration phase, computed as the absolute volume difference between two consecutive breathing onsets, is also shown below the respiration maneuver in FIG. 14.

F. Instantaneous Respiration Rate Estimation Using Smartphone Camera Signal

To estimate IRR from the smartphone-acquired chest movement signal, a time-varying spectral technique was used. The smoothed pseudo Wigner-Ville distribution (SPWVD) time-frequency representation (TFR) was employed. A TFR is a function that simultaneously describes the energy density of a signal in the time and frequency domains, allowing one to analyze which frequencies of a signal under study are present at a certain time (L. Cohen, “Time-frequency distributions-a review,” Proc. IEEE, vol. 77, no. 7, pp. 941-981, July 1989.). Then, TFR analysis is useful for analyzing signals whose frequency content varies over time, as is the case with respiration signals. Note that the use of a simple peak detector would be an option for estimating the instantaneous respiratory rates. However, due to low sampling rates and not-well-defined breathing peaks, all simple peak detectors result in less accurate respiratory rate estimation than do time-varying spectral approaches. The Wigner-Ville distribution (WVD) belongs to the Cohen's class of bilinear time-frequency representations; it possesses several interesting properties, and in particular provides the highest time-frequency resolution. However, the main limitation of the WVD is the presence of cross-terms that obscure its readability. Several techniques have been proposed to reduce the number of cross-terms of the WVD; however, there is a tradeoff between the amount of cross-term interference and the time-frequency resolution. The spectrogram is one such attempt, a joint time-frequency smoothing window is applied and hence the performance in one direction is enhanced at the expense of degrading the performance in the other. In contrast, the SPWVD employs independent time and frequency smoothing windows (W. Martin and P. Flandrin, “Wigner-Ville spectral analysis of nonstationary processes,” IEEE Trans. Acoust. Speech Signal Process., vol. 33, no. 6, pp. 1461-1470, December 1985), as given by

$$SPWVD ( t , f ) = {\int }_{- ∞}^{∞} h ( τ ) {\int }_{- ∞}^{∞} g ( η - t ) \cdot s ( η + \frac{τ}{2} ) {s}^{*} ( η + \frac{τ}{2} ) d {ηe}^{- j2π f τ} d τ \qquad ( 11 )$$

where s(t) is the signal under analysis, g(·)is the time smoothing window, and h(·)is the frequency smoothing window in the time-domain (F. Hlawatsch, T. G. Manickam, R. L. Urbanke, and W. Jones, “Smoothed pseudo-Wigner distribution, Choi-Williams distribution, and cone-kernel representation: Ambiguity-domain analysis and experimental comparison,” Signal Process., vol. 43, no. 2, pp. 149-168, May 1995).

The SPWVD was applied to the volume and chest movement signals. The SPWVD was computed using NFFT=1024 frequency bins, a 2 second Hamming window as the time smoothing window, and a 5.12 second Hamming window as the frequency smoothing window. After computing, the SPWVD was normalized between [0-1]. The Welch modified periodogram was used to compute the spectrum of the whole maneuver in order to obtain the central or average respiration frequency as the maximum spectral peak. The periodogram was computed using 50% overlap, 512 frequency bins, and a Hamming window. Then, at each time instant the maximum peak around the central frequency was computed and the frequency at which that maximum occurs was regarded as the respiration frequency at that instant, so that a vector of instantaneous respiration frequency was returned from each SWPVD. The frequency vector extracted from the spirometer-based volume was regarded as the reference instantaneous respiration frequency and was compared against the frequency vector extracted from the corresponding smartphone-based chest movement signal. All instantaneous respiration frequencies were converted from hertz to breaths-per-minute (bpm) to obtain IRR. Note that the SPWVD is a well-known time-varying spectral approach, which can be implemented in a variety of programming languages including the ones used for the smartphone app development, Java.

Similar to tidal volume estimation, the performance of the IRR estimation using the smartphone-acquired chest movement signal was tested using three performance indices by considering the IRR from volume signal as reference: the root-mean-squared error RMSE, the normalized root-mean-squared error NRMSE, and the cross-correlation index ρ defined as follows

$$ρ = \frac{{\sum }_{i = 1}^{S} {IRR}_{spirometer} ( i ) \cdot {IRR}_{smartphone} ( i )}{\sqrt{{\sum }_{i = 1}^{S} {( {IRR}_{spirometer} ( i ) )}^{2} \cdot {\sum }_{i = 1}^{S} {( {IRR}_{smartphone} ( i ) )}^{2}}} \qquad ( 12 )$$

where IRRₛₚᵢᵣₒₘₑₜₑᵣ indicates the IRR obtained from the spirometer-acquired volume signal, IRRₛₘₐᵣₜₚₕₒₙₑ is the IRR estimated from smartphone-acquired chest movements, and S is the number of samples of the analyzed signal, i.e., time instants. RMSE and NRMSE were computed via (3) and (4), by replacing the V_{T} values at each breath-phase by the IRR values at each time instant. A flow chart of the IRR estimation stage is shown in FIG. 15C. III. Results

The smartphone-acquired chest movement signal showed temporal amplitude variation related to the volume from spirometer during the breathing maneuver as shown in FIG. 13 and more evidently in FIG. 14 after detrending. In the following subsections, the results in terms of tidal volume estimation and respiration rate estimation using this smartphone-acquired chest movement signal are presented. The distribution of the number of breathing cycles, average V_{T}, and average RR performed by the volunteers during the breathing maneuvers are shown in Table I. As can be seen, the maneuvers included a wide range of breathing cycles, rates and depths. Table II presents relevant information for all subjects concerning their biometrics, the corresponding calibration coefficients used for V_{T} estimation, and the comparison of the V_{T} and IRR smartphone-based estimates to the ones obtained from the reference signal from spirometry.

TABLE I
DISTRIBUTION OF BREATHING CYCLES, TIDAL
VOLUME AND RESPIRATION RATE MEASURED
BY SPIROMETER DURING BREATHING
MANEUVERS (N = 15 SUBJECTS).

| Parameter | | Min | Max | Average |
|---|---|---|---|---|
| Breathing | [cycles] | 16 | 51 | 31.40 ± 10.25 |
| cycles | | | | |
| Maneuver tidal | [L] | 0.24 ± 0.11 | 3.11 ± 0.67 | 1.32 ± 0.26 |
| volume | | | | |
| Maneuver | [bpm] | 11.08 ± 3.69 | 35.45 ± 13.04 | 17.12 ± 5.28 |
| respiration | | | | |
| rate | | | | |

Values presented as mean ± standard deviation

TABLE II
INFORMATION RELATED TO THE BIOMETRICS, BREATHING MANEUVER, CALIBRATION MODEL,
AND PERFORMANCE OF THE SMARTPHONE-BASED TIDAL VOLUME AND INSTANTANEOUS RESPIRATION
RATE ESTIMATES IN COMPARISON TO THE REFERENCE SIGNAL FROM SPIROMETER FOR
EACH OF THE N = 15 SUBJECTS.

| | | | | | | | Calbration | | | V_{T} | | IRR | |
|---|---|---|---|---|---|---|---|---|---|---|---|---|---|
| | | | | | Body | | parameters | | | estimation | | estimation | |
| | | | | | mass | | for V_{T} | | | errors | | errors | |
| Subject | | Age | Weight | Height | index | Breating | estimation | | | RMSE | NRMSE | RMSE | NRMSE |
| No. | Gender | [years] | [kg] | [m] | [kg/m²] | cycles | m | b | r³ | [L] | [%] | [bpm] | [%] |
| 1 | M | 32 | 75 | 1.63 | 28.23 | 51 | 0.148 | 0.093 | 0.994 | 0.119 | 11.105 | 0.361 | 1.477 |
| 2 | M | 35 | 70 | 1.70 | 24.22 | 36 | 0.138 | 0.200 | 0.983 | 0.126 | 11.007 | 0.312 | 1.426 |
| 3 | M | 32 | 60 | 1.79 | 18.73 | 36 | 0.134 | 0.483 | 0.944 | 0.281 | 19.691 | 0.422 | 1.731 |
| 4 | M | 24 | 82 | 1.72 | 27.72 | 40 | 0.147 | 0.173 | 0.980 | 0.484 | 26.996 | 0.295 | 1.440 |
| 5 | F | 35 | 60 | 1.65 | 22.04 | 37 | 0.140 | 0.199 | 0.966 | 0.286 | 21.526 | 0.349 | 1.620 |
| 6 | M | 52 | 70 | 1.73 | 23.39 | 37 | 0.295 | −0.131 | 0.924 | 0.261 | 16.038 | 0.288 | 1.898 |
| 7 | M | 26 | 77 | 1.77 | 24.58 | 31 | 0.076 | 0.206 | 0.921 | 0.064 | 9.371 | 0.343 | 1.845 |
| 8 | M | 25 | 70 | 1.78 | 22.09 | 46 | 0.114 | 0.166 | 0.966 | 0.127 | 10.293 | 0.336 | 2.200 |
| 9 | M | 19 | 73 | 1.83 | 21.80 | 23 | 0.086 | 0.341 | 0.933 | 0.095 | 9.176 | 0.321 | 2.167 |
| 10 | M | 19 | 62 | 1.79 | 19.35 | 17 | 0.071 | 0.078 | 0.975 | 0.137 | 13.861 | 0.472 | 5.177 |
| 11 | M | 19 | 64 | 1.80 | 19.75 | 21 | 0.068 | 0.161 | 0.984 | 0.189 | 21.134 | 0.437 | 4.899 |
| 12 | M | 22 | 69 | 1.78 | 21.78 | 29 | 0.110 | 0.588 | 0.867 | 0.141 | 13.749 | 0.310 | 1.462 |
| 13 | M | 46 | 98 | 1.80 | 30.25 | 16 | 0.360 | 0.135 | 0.938 | 0.128 | 13.995 | 0.975 | 12.543 |
| 14 | M | 36 | 62 | 1.76 | 20.02 | 24 | 0.132 | −0.231 | 0.850 | 0.134 | 13.628 | 0.621 | 4.861 |
| 15 | M | 21 | 88 | 1.82 | 26.37 | 23 | 0.092 | −0.224 | 0.935 | 0.139 | 13.400 | 0.386 | 2.317 |

| | | | | | | | | | | | | |
|---|---|---|---|---|---|---|---|---|---|---|---|---|
| Mean | 28.73 | 78.14 | 1.76 | 23.37 | 31.48 | 0.141 | 0.149 | 8.952 | 0.182 | 14.998 | 0.414 | 3.831 |
| S.D | 9.27 | 19.83 | 8.06 | 3.51 | 10.25 | 0.082 | 0.227 | 8.043 | 0.107 | 5.171 | 0.176 | 2.873 |

### A. Tidal Volume Estimation Using Smartphone Camera Signal

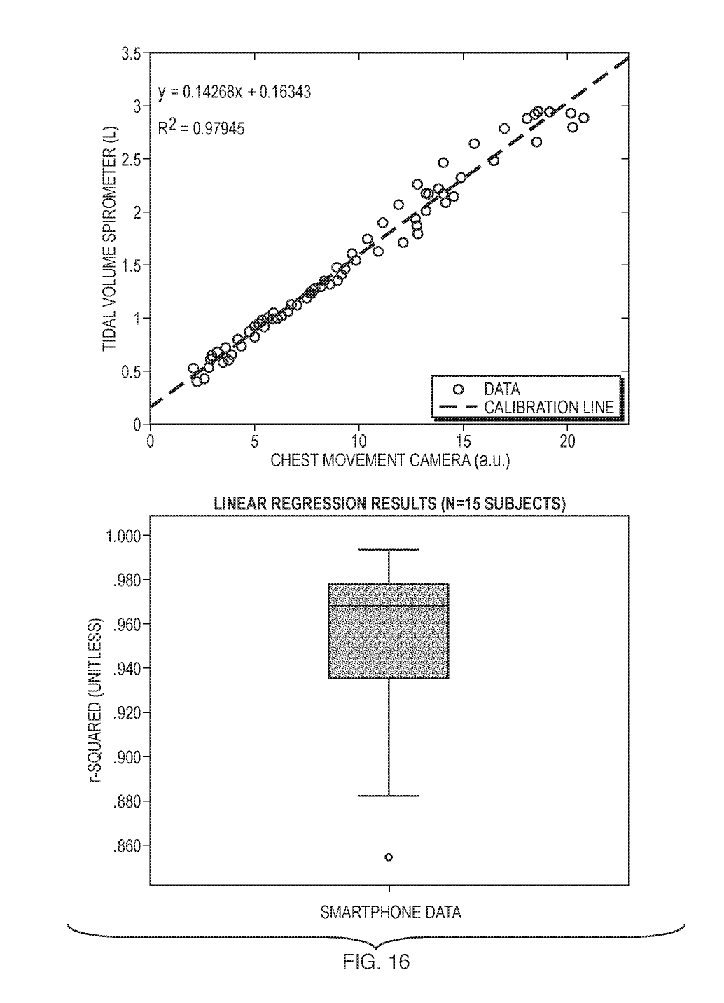

FIG. 16 shows the relationship between the absolute peak-to-peak amplitude of chest movement acquired with the smartphone and the reference tidal volume acquired with the spirometer for each breath phase of the maneuver performed by one subject. As shown in this figure, the amplitude differences of smartphone-based chest movement signals linearly correlate to reference V_{T} from the spirometer. The regression parameter r² between the absolute peak-to-peak amplitude of chest movement and reference tidal volume was computed for all breath-phases of each subject (r²=0.951±0.042, mean±SD). The corresponding boxplot for all subjects is also shown in FIG. 16. Strong linear relationship (r²>0.9) was found between the smartphone-based estimates and the reference tidal volume from the spirometer, as tested via a one-sample Wilcoxon signed rank test (p=6.41×10⁻⁴) after the normality assumption did not hold (one-sample Kolmogorov-Smirnov test, p=0.002).

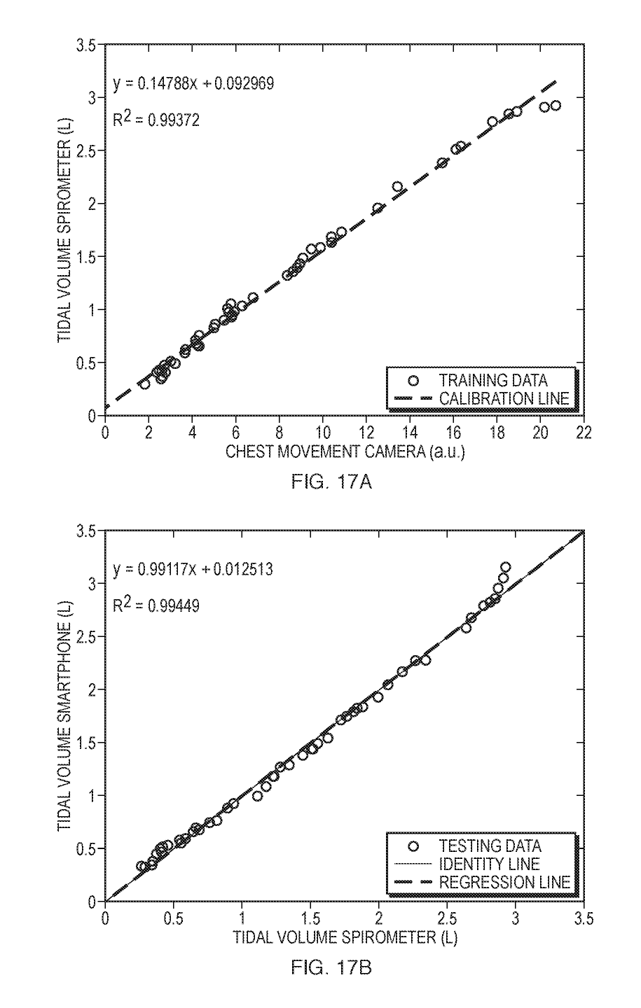

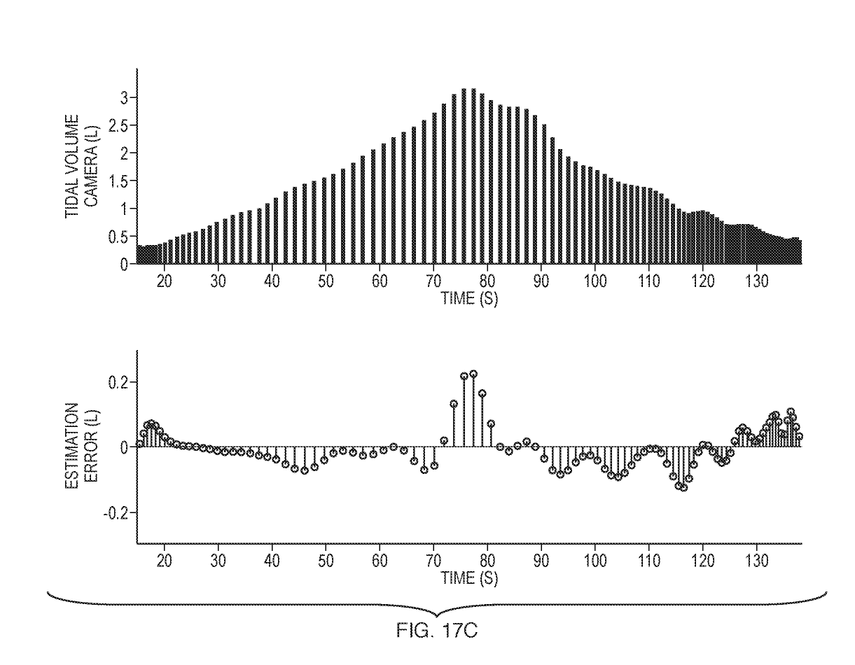

An example of the V_{T} estimation procedure from smartphone-acquired data is shown in FIG. 17A-C. From top to bottom, the first plots FIG. 17A-B correspond to the calibration process using the training data set (FIG. 17A), and the testing process using the remaining randomly-selected breath-phase data points (FIG. 17B), respectively. The calibration parameters were computed via least-squares linear regression. FIG. 17C shows the corresponding smartphone-based V_{T} estimates, after using the calibration parameters, for each breath phase of the maneuver of one subject. The lower panel of FIG. 17C shows the corresponding error differences with respect to the reference V_{T} from spirometry.

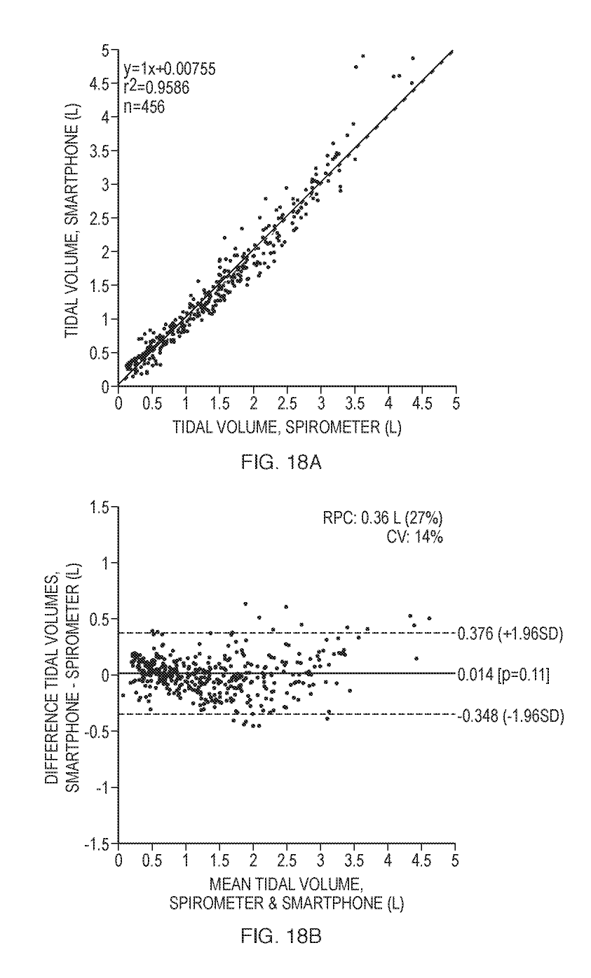

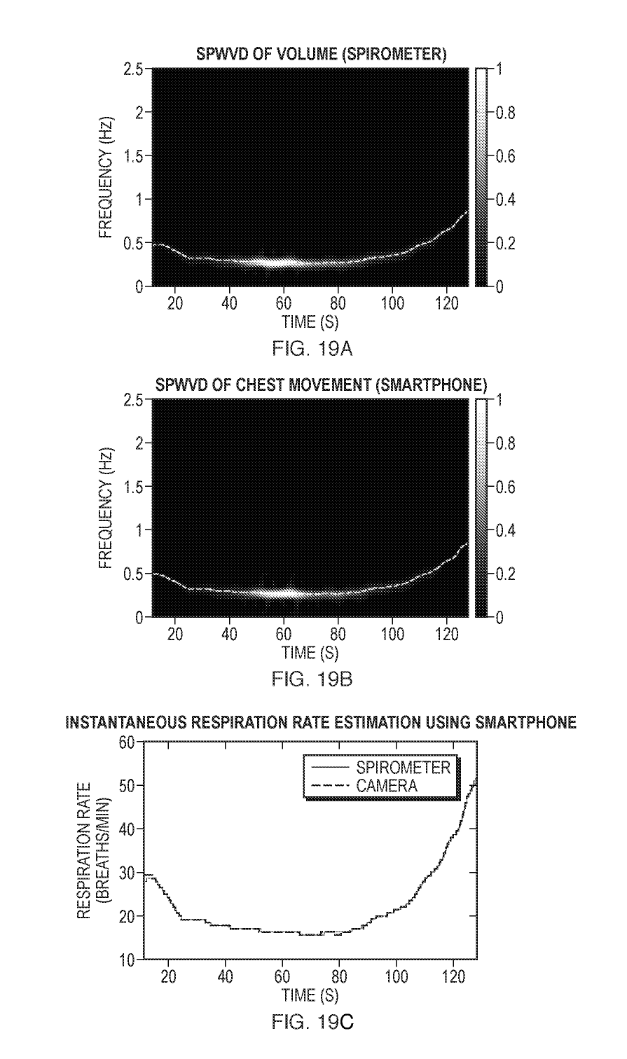

The performance indices for smartphone-based V_{T} estimation are presented in Table III for the testing data set of all the volunteers, using the spirometer measurements as reference. The linear regression results shown in FIG. 17A-C, for one subject, hold for all subjects, as shown in FIG. 18A, when a linear regression was applied to all the tidal volume estimates from all volunteers. FIG. 18B also presents the corresponding Bland-Altman plot.

FIG. 18A is a plot of linear regression results.

FIG. 18B is a Bland-Altman plot corresponding to FIG. 18A.

It was found that when calibrated on a subject-by-subject basis, the smartphone-based V_{T} estimation produced a bias of 0.014 liters and a standard deviation of 0.185 liters, however the bias was not found to be statistically significant from a zero bias. Accordingly, the 95% limits of agreements were −0.348 to 0.376 liters.

TABLE III
RESULTS OF TIDAL VOLUME ESTIMATION USING
SMARTPHONE-ACQUIRED CHEST MOVEMENT
SIGNALS COMPARED TO THE REFERENCE
VOLUME FROM THE SPIROMETER
(N = 15 SUBJECTS).

| | |
|---|---|
| Parameter | Values |

| | | |
|---|---|---|
| r² | [unitless] | 0.961 ± 0.026 |
| RMSE | [L] | 0.182 ± 0.107 |
| NRMSE | [%] | 14.998 ± 5.171 |

Values presented as mean ± standard deviation

### B. Instantaneous Respiration Rate Estimation Using Smartphone Camera Signal

FIGS. 19A-C show an example of IRR estimation via the SPWVD technique applied to volume from a spirometer and chest movements from the smartphone for the respiration maneuver of one subject. The superimposed white dashed curve indicates the frequency at which the maximum energy of the SPWVD occurs at each time instant. Side-by-side comparison of the extracted IRR from spirometer and smartphone signals is also presented. Observe that the subject was breathing at a faster pace than normal in order to account for the lower tidal volumes at the beginning and the end of the maneuver.

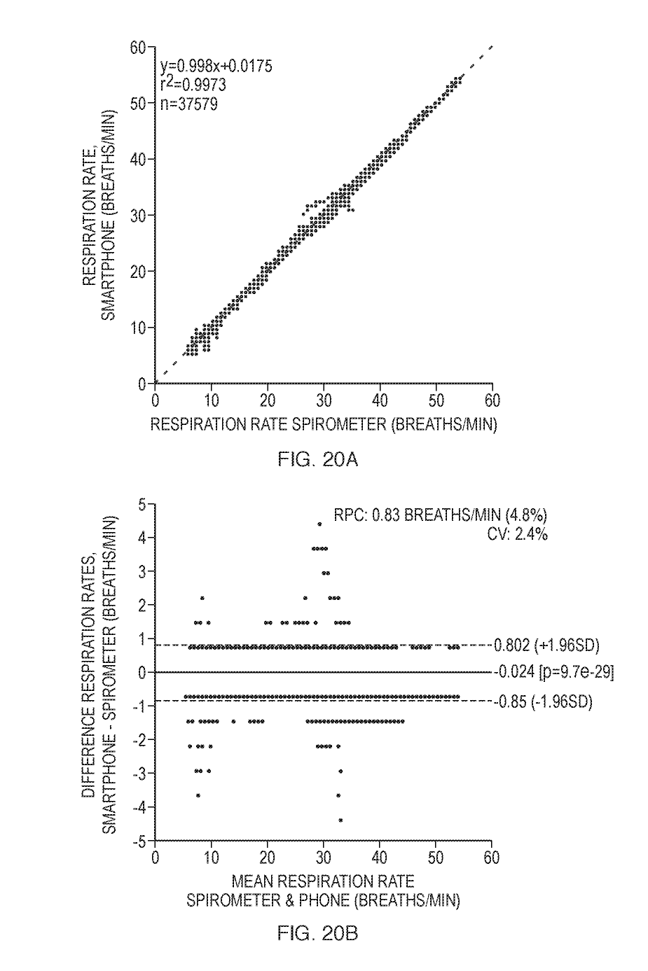

Table IV presents the performance indices of smartphone-based IRR estimation for all the subjects, using the spirometer values as reference. High cross-correlation coefficients were found between the IRR smartphone-based estimates and volume from spirometer. FIG. 20A reflects this high correlation as shown by the regression line parameters (r²=0.9973). The corresponding Bland-Altman plot is also presented in FIG. 20B. Compared to the spirometer, the bias±standard deviation and the 95% limits of agreement were −0.024±0.421 bpm and −0.850 to 0.802 bpm, respectively. Note that in this Bland-Altman plot, the IRR differences distribute at regular intervals given by the width of the frequency bins used in the calculation of the FFT during the time-frequency analysis,

$$Δ = \frac{fs / 2}{NFFT} = 0.0122 Hz$$

equivalent to Δ=0.7324 bpm .

IV. Discussion and Conclusions

According to some embodiments, a smartphone-based respiration monitoring system for both instantaneous respiration rate estimation and tidal volume estimation a method according to embodiments disclosed herein that tracks chest movements directly from a smartphone's camera. The HTC One M8 Android® smartphone was used in this study and the method was implemented in this device so that recordings of the chest movement signals were made directly on the phone. Together with this smartphone signal, airflow and volume signals were recorded with a spirometer and the latter was used as reference for IRR and V_{T} estimation. Recordings from fifteen healthy volunteers were obtained in a regular dry lab illuminated with fluorescent light while the volunteers were standing still and breathing at tidal volumes ranging from 300 mL to 3 L. Volunteers wore clothes with different colors and patterns. The developed method can still detect the chest movements even if single color clothes are worn.

TABLE IV
RESULTS OF THE INSTANTANEOUS RESPIRATION RATE
ESTIMATION USING SMARTPHONE-ACQUIRED CHEST
MOVEMENT SIGNAL COMPARED TO VOLUME SIGNAL
FROM SPIROMETER (N = 15 SUBJECTS).

| | |
|---|---|
| Parameter | Values |

| | | |
|---|---|---|
| ρ | [unitless] | 0.9992 ± 0.0019 |
| RMSE | [bpm] | 0.414 ± 0.178 |
| NRMSE | [%] | 3.031 ± 2.873 |

Values presented as mean ± standard deviation

There have been several efforts to develop monitors that provide information about breathing status via optical approaches (F. Zhao, M. Li, Y. Qian, and J. Z. Tsien, “Remote Measurements of Heart and Respiration Rates for Telemedicine,” PLoS ONE, vol. 8, no. 10, p. e71384, October 2013; M. Bartula, T. Tigges, and J. Muehlsteff, “Camera-based system for contactless monitoring of respiration,” in 2013 35th Annual International Conference of the IEEE Engineering in Medicine and Biology Society (EMBC), 2013, pp. 2672-2675; S. J. Cala, C. M. Kenyon, G. Ferrigno, P. Carnevali, A. Aliverti, A. Pedotti, P. T. Macklem, and D. F. Rochester, “Chest wall and lung volume estimation by optical reflectance motion analysis,” J. Appl. Physiol., vol. 81, no. 6, pp. 2680-2689, December 1996; M.-Z. Poh, D. J. McDuff, and R. W. Picard, “Advancements in Noncontact, Multiparameter Physiological Measurements Using a Webcam,” IEEE Trans. Biomed. Eng., vol. 58, no. 1, pp. 7-11, January 2011; D. Shao, Y. Yang, C. Liu, F. Tsow, H. Yu, and N. Tao, “Noncontact Monitoring Breathing Pattern, Exhalation Flow Rate and Pulse Transit Time,” IEEE Trans. Biomed. Eng., vol. 61, no. 11, pp. 2760-2767, November 2014; L. Tarassenko, M. Villarroel, A. Guazzi, J. Jorge, D. A. Clifton, and C. Pugh, “Non-contact video-based vital sign monitoring using ambient light and auto-regressive models,” Physiol. Meas., vol. 35, no. 5, p. 807, 2014; H.-Y. Wu, M. Rubinstein, E. Shih, J. Guttag, F. Durand, and W. Freeman, “Eulerian Video Magnification for Revealing Subtle Changes in the World,” ACM Trans Graph, vol. 31, no. 4, pp. 65:1-65:8, July 2012), most of them monitoring only average RR.

A method according to embodiments disclosed herein that is able to track chest movements directly on a smartphone was implemented and promising results were found in terms of average RR estimation. That study provided motivation to explore whether information beyond the average RR can be obtained from the smartphone-acquired chest movement signal. In particular, it appeared that the smartphone app provided a signal whose peak-to-peak amplitude may be an indicator of the tidal volume of the volunteers. This hypothesis was corroborated as exemplified in the recorded reference volume and chest movement signals, especially after detrending via EMD to remove existing drift in both signals.

The correlation of the peak-to-peak amplitude of smartphone-acquired signals with the corresponding tidal volume signal acquired from a spirometer was also analyzed. It was found that a strong correlation existed between the peak-to-peak amplitude of chest movement signals and tidal volume from the spirometer (r²=0.951±0.042, mean±SD). Given these correlation results, for each subject 50% of the data points were randomly selected for training the linear model during the calibration process, and the remaining 50% of the data for testing the tidal volume estimation based on the computed model. Once calibrated on an individual basis using the reference volume signal, the chest movement amplitude differences were mapped at each breath-phase of the testing data set, it was found that an RMSE of 0.182±0.107 liters which corresponded to 14.998±5.171% when normalized to the mean value of the reference V_{T} of the testing data set of the maneuver. Overall, it was found that a linear regression model fitted well the calibrated peak-to-peak amplitude of smartphone signals for the task of V_{T} estimation (V_{Tsmartphone}=1.005·V_{Tspirometer}+0.008). No statistically-significant bias was found in the V_{T} estimation using smartphones and the 95% limits of agreement were −0.348 to 0.376 liters. At this point it is difficult to state if this error estimate in tidal volume is acceptable for home monitoring use.

Other popular methods for tidal volume estimation suffer from even higher estimation errors, for example, respiratory inductance plethysmography (RIP), when calibrated according to the manual (which usually states that 10% error difference is acceptable), often has much higher errors. Others have reported similar findings with respect to errors, e.g., reference (K. P. Cohen, W. M. Ladd, D. M. Beams, W. S. Sheers, R. G. Radwin, W. J. Tompkins, and J. G. Webster, “Comparison of impedance and inductance ventilation sensors on adults during breathing, motion, and simulated airway obstruction,” IEEE Trans. Biomed. Eng., vol. 44, no. 7, pp. 555-566, July 1997.) found a bias and 95% limits of agreement in RIP sensors of approximately 0.4 L, and −0.3 to 1.1 L for a breathing range of 360 mL to 3.5 L; however, the estimation error using RIP is even higher than the method according to embodiments disclosed herein using a smartphone's video camera.

By taking advantage of the high correlation between detrended smartphone signals and volume from the spirometer, using the smartphone signal for the task of RR estimation at each time instant was analyzed. Due to the time-varying characteristics of the signals, the smoothed pseudo Wigner-Ville distribution was employed. High correlation between the smartphone-based IRR estimates and the spirometer-based values (r²=0.9992±0.0019) was found. An RMSE of 0.414±0.178 bpm was found which corresponds to an NRMSE of 3.031±2.783%. The linear relationship between IRR estimated from the smartphone and IRR from reference volume was IRRₛₘₐᵣₜₚₕₒₙₑ=0.9980·IRRₛₚᵢᵣₒₘₑₜₑᵣ+0.0175. The 95% limits of agreement ranged from −0.850 to 0.802 bpm, while there was a statistically-significant bias of −0.024 bpm. Other studies have reported the estimation of respiratory rate using noncontact optical approaches, e.g., in M. Bartula, T. Tigges, and J. Muehlsteff, “Camera-based system for contactless monitoring of respiration,” in 2013 35th Annual International Conference of the IEEE Engineering in Medicine and Biology Society (EMBC), 2013, pp. 2672-2675) the bias and standard deviation were found to be 0.19 bpm and 2.46 bpm, respectively, in the range of approximately 10-70 bpm; in (M.-Z. Poh, D. J. McDuff, and R. W. Picard, “Advancements in Noncontact, Multiparameter Physiological Measurements Using a Webcam,” IEEE Trans. Biomed. Eng., vol. 58, no. 1, pp. 7-11, January 2011) the RMSE, bias, and standard deviation were 1.28 bpm, 0.12 bpm, and 1.33 bpm, respectively, in the range of approximately 10-22 bpm; in (D. Shao, Y. Yang, C. Liu, F. Tsow, H. Yu, and N. Tao, “Noncontact Monitoring Breathing Pattern, Exhalation Flow Rate and Pulse Transit Time,” IEEE Trans. Biomed. Eng., vol. 61, no. 11, pp. 2760-2767, November 2014) the RMSE, bias and 95% limits of agreement were 1.20 bpm, 0.02 bpm, -−2.40 to 2.45 bpm, respectively, in the range of approximately 10-24 bpm; while in (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press) the RMSE, bias, and 95% limits of agreement were found to be 0.09 bpm, −0.02 bpm, and −1.69 to 1.65 bpm, respectively, in the range of approximately 7-24 bpm. Interestingly, the results reported in (Reyes, B. A.; Reljin, N.; Kong, Y.; Nam, Y.; Chon, K. H. Tidal Volume and Instantaneous Respiration Rate Estimation using a Volumetric Surrogate Signal Acquired via a Smartphone Camera. IEEE J. Biomed. Health Inform. 2016, In Press) during night conditions outperformed those mentioned in the several sentence above during daylight conditions. Although a straightforward comparison is not possible due to the differences in the measurement devices and the noncontact distance ranges tested, in general, results disclosed herein indicate that noncontact optical monitoring of respiratory rate based on smartphones performs as well as, if not better than, the aforementioned studies.

The recording of the breathing maneuvers was performed while the subjects were standing still, i.e., the subjects were instructed not to move. As found in other noncontact optical approaches, the main challenge arises from motion artifacts, especially when the dynamics of both the volumetric surrogate signal obtained from the chest wall movements and the motion artifacts have similar low frequency ranges (<2 Hz). Hence, it is expected that motion artifacts deteriorate the performance of the smartphone-based breathing estimates. Implementation of body tracking and artifact removal schemes similar to those reported in the literature to improve respiratory rate estimation (D. Shao, Y. Yang, C. Liu, F. Tsow, H. Yu, and N. Tao, “Noncontact Monitoring Breathing Pattern, Exhalation Flow Rate and Pulse Transit Time,” IEEE Trans. Biomed. Eng., vol. 61, no. 11, pp. 2760-2767, November 2014), (Y. Sun, S. Hu, V. Azorin-Peris, S. Greenwald, J. Chambers, and Y. Zhu, “Motion-compensated noncontact imaging photoplethysmography to monitor cardiorespiratory status during exercise,” J. Biomed. Opt., vol. 16, no. 7, pp. 077010-077010, 2011) are expected to reduce the effect of body motion not related to the breathing maneuver. Implementation and testing of such methods in the smartphone for respiratory monitoring, especially for the task of tidal volume estimation, may be useful.

Another major challenge is the variation of the ambient illumination at different times of the day due to fluctuations in the amount of sunlight, for example. The experiments presented herein were performed at different times of the day and while the main illumination source came from the ceiling fluorescent lamps, the window shades of the laboratory were kept open or closed according to the needs of its users. Despite that, it was noticed that these variations disturbed the acquisition of the volumetric surrogate signal, perhaps due to the dominance of the fluorescent source.

Classically, chest wall movements are attributed to two mechanical degrees of freedom due to contributions from rib cage and abdomen, which can be used to estimate tidal volume (K. Konno and J. Mead, “Measurement of the separate volume changes of rib cage and abdomen during breathing,” J. Appl. Physiol., vol. 22, no. 3, pp. 407-423, March 1967). Although 1D or 2D displacements of these two compartments account for the majority of tidal volume, the algorithm ignores systematic effects of rib cage distortions (S. J. Cala, C. M. Kenyon, G. Ferrigno, P. Carnevali, A. Aliverti, A. Pedotti, P. T. Macklem, and D. F. Rochester, “Chest wall and lung volume estimation by optical reflectance motion analysis,” J. Appl. Physiol., vol. 81, no. 6, pp. 2680-2689, Dec. 1996). Herein, the chest movement signal used as volume surrogate was extracted from an image's rectangular area centered on the anterior chest wall portion of the volunteer that visually provided the most dominant displacements while breathing. Accordingly, embodiments disclosed herein may ignore those small contributions due to rib cage distortions and only constructs the chest movement signal from the chest wall displacements monitored by the camera.

It is expected that postural changes and airway obstruction impact the performance of the estimates, as has been found in other breathing monitor techniques (T. M. Baird and M. R. Neuman, “Effect of infant position on breath amplitude measured by transthoracic impedance and strain gauges,” Pediatr. Pulmonol., vol. 10, no. 1, pp. 52-56, 1991; M. J. Tobin, S. M. Guenther, W. Perez, and M. J. Mador, “Accuracy of the respiratory inductive plethysmograph during loaded breathing,” J Appl Physiol, vol. 62, no. 2, pp. 497-505, 1987). Postural changes can modify the contribution of the rib cage and abdomen compartments to tidal volume. A decreased rib cage excursion and an increased abdominal excursion have been found in the supine position compared to the sitting or standing postures (V. P. Vellody, M. Nassery, W. S. Druz, and J. T. Sharp, “Effects of body position change on thoracoabdominal motion,” J. Appl. Physiol., vol. 45, no. 4, pp. 581-589, Oct. 1978; W. S. Druz and J. T. Sharp, “Activity of respiratory muscles in upright and recumbent humans,” J. Appl. Physiol., vol. 51, no. 6, pp. 1552-1561, December 1981). Accordingly, another area of the thorax may be used to provide a stronger surrogate signal when monitoring breathing in the supine position.

The subjects wore fitted clothes during the experiments. As pointed out by other researchers, if the clothes are not tight enough to the subject's body a weak breathing-related signal might be obtained using the noncontact optical monitoring approach. Note that this is also the case in other respiratory monitoring methods based on chest wall displacements, like inductance plethysmography, where the sensors are recommended to be worn over bare skin or tight clothes. Observe that in general, the noncontact optical approach looks for changes in the light intensity due to the modification of the path length caused by breathing displacements of the chest wall, and is not limited to movements of clothing features. However, a study to analyze the effect of wearing loose-fitting clothes may be useful. Finally, note that to estimate tidal volume via the smartphone's camera, the measurement conditions should match those during which calibration was performed.

Although it was found that a linear model fit well between peak-to-peak amplitude of chest movement signals from a smartphone and tidal volume from a spirometer, so that it can be used to calibrate the smartphone measurements to obtain tidal volume on an individual basis, calibration may be performed prior to acquisition if the subject's chest wall position monitored by the smartphone's camera displaces with respect to the one used for calibration. Other tidal volume estimation techniques suffer similar issues, e.g., displacement of elastic belts wrapped around the rib cage and abdomen from the position employed when calibration was performed deteriorates the performance of the measurements in inductance plethysmography.

Several monitoring techniques for breathing status in clinical and research settings currently exist. Embodiments disclosed herein enable developing of an inexpensive and mobile respiratory monitoring system that can be translated outside research settings for on-demand health applications. By taking advantage of their ubiquity, smartphone-based systems could aid in the monitoring of breathing status of the general population, where this general practice remains unclear if it is considered that these parameters are not always recorded on a daily basis even in clinical settings. The results obtained in herein point out the feasibility of developing a mobile system being able to provide information about instantaneous respiration rate and tidal volume when calibrated on an individual basis. It is foreseen that when calibration is not possible to be performed, this smartphone approach could still be used as a qualitative indicator of changes in tidal volume due to the high correlation between the chest movement signal and tidal volume that reflects the major contribution of chest wall displacements to tidal volume. To this end, embodiments disclosed herein may take an initial step towards the estimation of V_{T} from a surrogate signal obtained with a smartphone. Conclusions about the robustness in terms of measurement conditions such as gender, body mass index or lighting conditions cannot be made given the small sample size and conditions tested and may be explored in future studies.

As disclosed above, an efficient, automated and easy-to-use calibration procedure that can be performed with an incentive spirometer (IS) may be implemented. This is a low-cost off-the-shelf device which has the potential to be used in non-clinical settings. Briefly, by taking advantage of the high linear relationship between smartphone measurements and tidal volume, a calibration model may be computed while breathing at only two reference volume points through the IS. Embodiments disclosed herein enable a fast, automated and easy-to-perform calibration procedure with wireless remote controlling capabilities. It should be understood that while methods disclosed herein may have been developed for Android®, the methods may be applied to any suitable operating system.

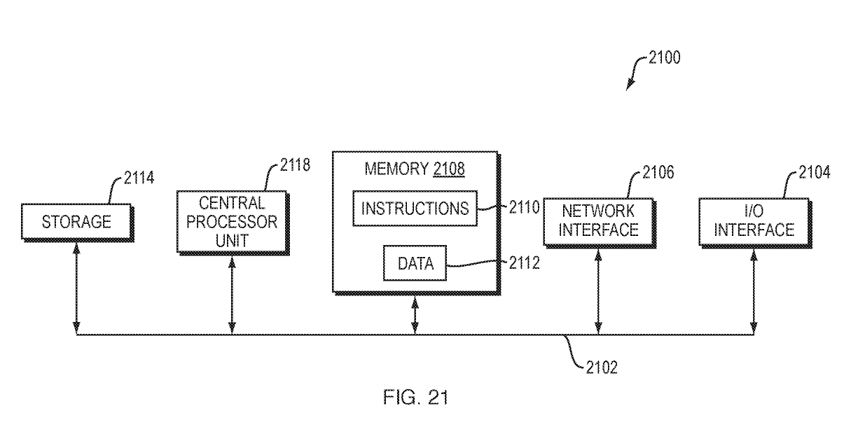

FIG. 21 is a block diagram of an example of the internal structure of a computer 2100 in which various embodiments of the present disclosure may be implemented. The computer 2100 contains a system bus 2102, where a bus is a set of hardware lines used for data transfer among the components of a computer or processing system. The system bus 2102 is essentially a shared conduit that connects different elements of a computer system (e.g., processor, disk storage, memory, input/output ports, network ports, etc.) that enables the transfer of information between the elements. Coupled to the system bus 2102 is an I/O device interface 2404 for connecting various input and output devices (e.g., keyboard, mouse, displays, printers, speakers, etc.) to the computer 2100. A network interface 2106 allows the computer 2100 to connect to various other devices attached to a network. Memory 2108 provides volatile storage for computer software instructions 2110 and data 2112 that may be used to implement embodiments of the present disclosure. Disk storage 2114 provides non-volatile storage for computer software instructions 2110 and data 2112 that may be used to implement embodiments of the present disclosure. A central processor unit 2118 is also coupled to the system bus 2102 and provides for the execution of computer instructions.

Further example embodiments disclosed herein may be configured using a computer program product; for example, controls may be programmed in software for implementing example embodiments. Further example embodiments may include a non-transitory computer-readable medium containing instructions that may be executed by a processor, and, when loaded and executed, cause the processor to complete methods described herein. It should be understood that elements of the block and flow diagrams may be implemented in software, hardware, such as via one or more arrangements of circuitry of FIG. 21, disclosed above, or equivalents thereof, firmware, a combination thereof, or other similar implementation determined in the future. In addition, the elements of the block and flow diagrams described herein may be combined or divided in any manner in software, hardware, or firmware. If implemented in software, the software may be written in any language that can support the example embodiments disclosed herein. The software may be stored in any form of computer readable medium, such as random access memory (RAM), read only memory (ROM), compact disk read-only memory (CD-ROM), and so forth. In operation, a general purpose or application-specific processor or processing core loads and executes software in a manner well understood in the art. It should be understood further that the block and flow diagrams may include more or fewer elements, be arranged or oriented differently, or be represented differently. It should be understood that implementation may dictate the block, flow, and/or network diagrams and the number of block and flow diagrams illustrating the execution of embodiments disclosed herein.

The teachings of all patents, published applications and references cited herein are incorporated by reference in their entirety.

While this invention has been particularly shown and described with references to example embodiments thereof, it will be understood by those skilled in the art that various changes in form and details may be made therein without departing from the scope of the invention encompassed by the appended claims.

What is claimed is:

1. A device for monitoring breathing, the device comprising a processor, the processor configured to:
compute a calibration signal from a first sequence of images of a user's chest to produce a calibration model, the calibration signal representative of movement of the user's chest during a first time period during which the user is using an incentive spirometer, the first sequence of images corresponding to the first time period; and
employ the calibration model to produce a breathing information estimate about the user's breathing from a second sequence of images of the user's chest corresponding to a second time period during which the user is not using the incentive spirometer.

2. The device of claim 1, wherein the breathing information estimate includes a representation of tidal volume, respiratory rate, or instantaneous respiratory rate, or a combination thereof.

3. The device of claim 1, wherein the device is a smartphone that includes:
an integrated camera configurable to capture the first sequence of images and the second sequence of images; and
the processor.

4. The device of claim 2, wherein the smartphone further includes:
a user interface; and
wherein the processor is further configured to output a representation of the breathing information estimate via the user interface.

5. The device of claim 2, wherein the smartphone further includes:
a user interface; and
wherein the processor is further configured to determine the first time period and the second time period based on interactions with the user interface via the user interface.

6. The device of claim 2, wherein the smartphone further includes:
a network interface; and
wherein the processor is further configured to output a representation of the breathing information estimate via the network interface.

7. The user device of claim 2, wherein the smartphone further includes:
a hardware interface configured to detect a usage signal, the usage signal representing usage of the incentive spirometer; and
wherein the processor is further configured to determine the first and second time periods based on detection of the usage signal.

8. The device of claim 1, wherein the device is a network server.

9. The device of claim 8, wherein the network server includes a network interface and wherein:
the network server is configured to receive the first sequence of images and the second sequence of images via the network interface; and
the processor is further configured to output a representation of the breathing information estimate via the network interface.

10. The device of claim 1, wherein using the incentive spirometer includes inhaling through the incentive spirometer or exhaling through the incentive spirometer.

11. The device of claim 1, wherein:
the first time period includes at least two time periods during which the user is using the incentive spirometer;
the first sequence of images includes at least two sequences of images corresponding to the at least two time periods; and
the calibration signal is further representative of movement of the user's chest during the at least two time periods, and wherein the user achieves a different target level on the incentive spirometer during respective periods of the at least two time periods.

12. The device of claim 1, further comprising a camera configurable to capture the first sequence of images and the second sequence of images.

13. The device of claim 1, further comprising a user interface, wherein the processor is further configured to output a representation of the breathing information estimate via the user interface.

14. The device of claim 1, further comprising a network interface and wherein the processor is further configured to output a representation of the breathing information estimate via the network interface.

15. The device of claim 1, wherein the device is a component within a system, the system including:
the device; and
a camera configurable to capture the first sequence of images and the second sequence of images.

16. The device of claim 1, wherein the calibration model is a linear model.

17. A method for monitoring breathing, the method comprising:
computing a calibration signal from a first sequence of images of a user's chest to produce a calibration model, the calibration signal representative of movement of the user's chest during a first time period during which the user is using an incentive spirometer, the first sequence of images corresponding to the first time period; and
employing the calibration model to produce a breathing information estimate about the user's breathing from a second sequence of images of the user's chest corresponding to a second time period during which the user is not using the incentive spirometer.

18. The method of claim 17, wherein the breathing information estimate includes a representation of tidal volume, respiratory rate, or instantaneous respiratory rate, or a combination thereof.

19. The method of claim 17, further comprising capturing the first sequence of images and the second sequence of images by a camera.

20. The method of claim 17 further comprising outputting a representation of the breathing information estimate via a user interface or a network interface.

21. The method of claim 17, further comprising determining the first time period and the second time period based on interactions with a user via a user interface.

22. The method of claim 17, further comprising:
detecting a usage signal, the usage signal representing usage of the incentive spirometer; and
determining the first and second time periods based on the usage signal detected.

23. The method of claim 17, further comprising:
receiving the first sequence of images and the second sequence of images via a network interface; and
outputting a representation of the breathing information estimate via the network interface.

24. The method of claim 17, wherein using the incentive spirometer includes inhaling through the incentive spirometer or exhaling through the incentive spirometer.

25. The method of claim 17, wherein:
the first time period includes at least two time periods during which the user is using the incentive spirometer;
the first sequence of images includes at least two sequences of images corresponding to the at least two time periods; and
the calibration signal is further representative of movement of the user's chest during the at least two time periods, wherein the user achieves a different target level on the incentive spirometer during respective periods of the at least two time periods.

26. The method of claim 17, wherein the calibration model is a linear model.

27. A non-transitory computer-readable medium having encoded thereon a sequence of instructions which, when loaded and executed by a processor, causes the processor to monitor breathing by:
computing a calibration signal from a first sequence of images of a user's chest to produce a calibration model, the calibration signal representative of movement of the user's chest during a first time period during which the user is using an incentive spirometer, the first sequence of images corresponding to the first time period; and
employing the calibration model to produce a breathing information estimate about the user's breathing from a second sequence of images of the user's chest corresponding to a second time period during which the user is not using the incentive spirometer.
